(19) United States
(12) Patent Application Publication
(10) Pub. No.: US 2022/0048900 A1
(43) Pub. Date: Feb. 17, 2022

(54) AMORPHOUS SPARSENTAN COMPOSITIONS

(71) Applicant: Travere Therapuetics, Inc., San Diego, CA (US)

(72) Inventors: Dainius Macikenas, San Diego, CA (US); Kale Ruby, Encinitas, CA (US); James Francis Hulvat, Bend, OR (US); Xiangming Wu, Winchester, MA (US)

(21) Appl. No.: 17/415,518

(22) PCT Filed: Dec. 20, 2019
(86) PCT No.: PCT/US2019/068094

§ 371 Date: Jun. 17, 2021

Related U.S. Application Data
(60) Provisional application No. 62/783,947, filed on Dec. 21, 2018.

(51) Int. Cl. C07D 413/12; A61K 31/422; A61K 47/38; A61K 47/32

(52) U.S. Cl. CPC C07D 413/12; A61K 47/32; A61K 47/38; A61K 31/422

(57) ABSTRACT

An amorphous form of a compound having structure (I), or a pharmaceutically acceptable salt thereof, is provided. Such compounds may be used for the treatment of kidney diseases or disorders.

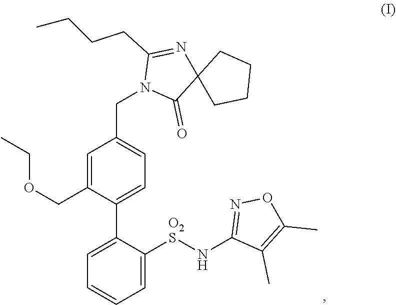 [SMILES: CCCCC1=NC2(CCCC2)C(=O)N1Cc1ccc(-c2ccccc2S(=O)(=O)Nc2noc(C)c2C)c(COCC)c1]

### BACKGROUND

The present disclosure relates to an amorphous form of sparsentan and solid formulations comprising the same, and their use in the treatment of kidney diseases or disorders.

Angiotensin II (AngII) and endothelin-I (ET-1) are two of the most potent endogenous vasoactive peptides currently known and are believed to play a role in controlling both vascular tone and pathological tissue remodeling associated with a variety of diseases, including diabetic nephropathy, heart failure, and chronic or persistently elevated blood pressure. Angiotensin receptor blockers (ARBs), which block the activity of AngII, have been used as a treatment for diabetic nephropathy, heart failure, and chronic or persistently elevated blood pressure. There is also a growing body of data that demonstrates the potential therapeutic benefits of ET receptor antagonists (ERAs) in blocking ET-1 activity. Additionally, AngII and ET-1 are believed to work together in blood pressure control and pathological tissue remodeling. For example, ARBs not only block the action of AngII at its receptor, but also limit the production of ET-1. Similarly, ERAs block ET-1 activity and inhibit the production of AngII. Consequently, simultaneously blocking AngII activity and ET-1 activity may offer better efficacy than blocking the activity of either molecule alone. In rat models of human chronic or persistently elevated blood pressure, the combination of an ARB and an ERA has been shown to result in a synergistic effect. Furthermore, although ARBs are the standard of care for patients with diabetic nephropathy, improved efficacy with the co-administration of an ERA has been reported in Phase 2 clinical development.

Sparsentan is a dual angiotensin and endothelin receptor antagonist in clinical development for the treatment of kidney diseases or disorders, some of which have no specific treatment or are associated with symptoms that are not entirely controlled by other therapies. Accordingly, there remains a need for forms and formulations of sparsentan that offer therapeutic benefits.

### BRIEF SUMMARY

In certain aspects, the present invention is directed to amorphous forms of a compound of structure (I):

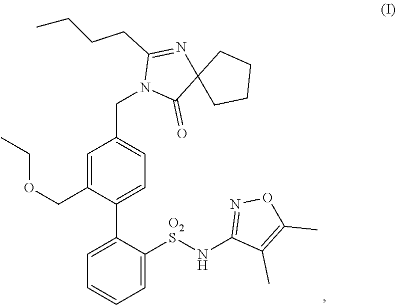 [SMILES: CCCCC1=NC2(CCCC2)C(=O)N1Cc1ccc(-c2ccccc2S(=O)(=O)Nc2noc(C)c2C)c(COCC)c1]

or a pharmaceutically acceptable salt thereof.

In certain other aspects, the present invention provides pharmaceutical compositions comprising an amorphous form of a compound of structure I, or pharmaceutically acceptable salt thereof, and a pharmaceutically acceptable excipient. In some embodiments, pharmaceutical compositions disclosed herein further comprise a polymer.

In certain other aspects, the present invention provides methods of treatment comprising administering to a subject the amorphous compounds or pharmaceutical compositions disclosed herein. Additionally, the present invention provides for the use of compounds and pharmaceutical compositions disclosed herein in treating diseases or disorders, and for their use in the manufacture of medicaments.

These and other aspects of the present invention will become apparent upon reference to the following detailed description.

### BRIEF DESCRIPTION OF THE DRAWINGS

FIG. 1. Powder X-ray diffraction (PXRD) diffractogram of amorphous sparsentan.

FIG. 2. Modulated differential scanning calorimetry (MDSC) thermogram of amorphous sparsentan.

FIG. 3. MDSC thermograms showing the glass transition temperature (Tg) of physical mixtures of sparsentan and various polymers at a weight ratio of 20:80: (1) 20:80 Sparsentan:Eudragit L100-55; (2) 20:80 Sparsentan:PVP-VA; (3) 20:80 Sparsentan:Affinisol 716; (4) 20:80 Sparsentan:Affinisol 912; (5) 20:80 Sparsentan:Affinisol 126; (6) 20:80 Sparsentan: HPMC HME; and (7) 20:80 Sparsentan:Soluplus.

FIG. 4. MDSC thermogram for crystalline sparsentan.

FIG. 5. MDSC thermograms showing the glass transition temperature (Tg) of spray dried dispersions of sparsentan and various polymers at a weight ratio of either 25:75 or 50:50: (1) 25:75 Sparsentan:PVP-VA; (2) 25:75 Sparsentan:HPMCAS-H; (3) 25:75 Sparsentan:Soluplus; (4) 25:75 Sparsentan:HPMC E3LV; (5) 50:50 Sparsentan:PVP-VA; (6) 50:50 Sparsentan: HPMCAS-H; (7) 50:50 Sparsentan:Soluplus.

FIG. 6. PXRD diffractograms of spray dried dispersions of sparsentan and various polymers at a weight ratio of either 25:75 or 50:50: (1) 25:75 Sparsentan:HPMC E3LV; (2) 25:75 Sparsentan:HPMCAS-H; (3) 25:75 Sparsentan: PVP-VA; (4) 25:75 Sparsentan:Soluplus; (5) 50:50 Sparsentan: PVP-VA; (6) 50:50 Sparsentan:Soluplus; (7) 50:50 Sparsentan:HPMC E3LV; and (8) 50:50 Sparsentan: HPMCAS-H.

FIG. 7. SEM images of spray dried sparsentan-polymer dispersions. Particles at 5,000× magnification. Upper panel, left to right: 25:75 Sparsentan: PVP-VA; 25:75 Sparsentan: HPMCAS-H; 25:75 Sparsentan:Soluplus; and 25:75 Sparsentan:HPMC E3LV. Lower panel, left to right: 50:50 Sparsentan: PVP-VA; 50:50 Sparsentan: HPMCAS-H; 50:50 Sparsentan:Soluplus; and 50:50 Sparsentan:HPMC E3LV.

FIG. 8. PXRD diffractograms of spray dried dispersions of sparsentan (without polymer); 80:20 Sparsentan:PVP-VA; and 65:35 Sparsentan:PVP-VA.

FIG. 9. MDSC thermograms showing the glass transition temperature (Tg) of spray dried dispersions of sparsentan (without polymer); 80:20 Sparsentan:PVP-VA; and 65:35 Sparsentan:PVP-VA.

FIG. 10. Mean (±SD) plasma concentration of sparsentan in male rats (3 animals/group), linear scale, following a single dose of sparsentan in different formulations by IV (1 mg/kg) or PO (20 and 60 mg/kg). F1: crystalline sparsentan; F2: 50:50 Sparsentan:PVP-VA SDD; F3: 50:50 Sparsentan:HPMC E3LV SDD; and F4: 50:50 Sparsentan:HPMCAS-H SDD.

FIG. 11. Mean (±SD) plasma concentration of sparsentan in male rats (3 animals/group), log 10 scale, following a single dose of sparsentan in different formulations by IV (1 mg/kg) or PO (20 and 60 mg/kg). F1: crystalline sparsentan; F2: 50:50 Sparsentan:PVP-VA SDD; F3: 50:50 Sparsentan:HPMC E3LV SDD; and F4: 50:50 Sparsentan:HPMCAS-H SDD.

FIG. 12. Mean (±SD) plasma concentration of sparsentan in male rats (3 animals/group), log 10 scale, following oral administration of a single dose of sparsentan in different formulations at 20 mg/kg. F1: crystalline sparsentan; F2: 50:50 Sparsentan:PVP-VA SDD; F3: 50:50 Sparsentan:HPMC E3LV SDD; and F4: 50:50 Sparsentan:HPMCAS-H SDD.

FIG. 13. Mean (±SD) plasma concentration of sparsentan in male rats (3 animals/group), log 10 scale, following oral administration of a single dose of sparsentan in different formulations at 60 mg/kg. F1: crystalline sparsentan; F2: 50:50 Sparsentan:PVP-VA SDD; F3: 50:50 Sparsentan:HPMC E3LV SDD; and F4: 50:50 Sparsentan:HPMCAS-H SDD.

FIG. 14. Comparison of plasma Cₘₐₓ in male rats (3 animals/group), following a single dose of sparsentan in different formulations by IV (1 mg/kg) and PO (20 and 60 mg/kg). F1: crystalline sparsentan; F2: 50:50 Sparsentan:PVP-VA SDD; F3: 50:50 Sparsentan:HPMC E3LV SDD; and F4: 50:50 Sparsentan:HPMCAS-H SDD.

FIG. 15. Comparison of plasma Cₘₐₓ/dose in male rats (3 animals/group), following a single dose of sparsentan in different formulations by IV (1 mg/kg) and PO (20 and 60 mg/kg). F1: crystalline sparsentan; F2: 50:50 Sparsentan:PVP-VA SDD; F3: 50:50 Sparsentan:HPMC E3LV SDD; and F4: 50:50 Sparsentan:HPMCAS-H SDD.

FIG. 16. Comparison of plasma AUC_{0-24 hr} in male rats (3 animals/group), following a single dose of sparsentan in different formulations by IV (1 mg/kg) and PO (20 and 60 mg/kg). F1: crystalline sparsentan; F2: 50:50 Sparsentan:PVP-VA SDD; F3: 50:50 Sparsentan:HPMC E3LV SDD; and F4: 50:50 Sparsentan:HPMCAS-H SDD.

FIG. 17. Comparison of plasma AUC_{0-24 hr}/dose in male rats (3 animals/group), following a single dose of sparsentan in different formulations by IV (1 mg/kg) and PO (20 and 60 mg/kg). F1: crystalline sparsentan; F2: 50:50 Sparsentan:PVP-VA SDD; F3: 50:50 Sparsentan:HPMC E3LV SDD; and F4: 50:50 Sparsentan:HPMCAS-H SDD.

FIG. 18. Mean pharmacokinetic parameters for male rats (3 animals/group), following a single dose of sparsentan in different formulations by IV (1 mg/kg) and PO (20 and 60 mg/kg). F1: crystalline sparsentan; F2: 50:50 Sparsentan:PVP-VA SDD; F3: 50:50 Sparsentan:HPMC E3LV SDD; and F4: 50:50 Sparsentan:HPMCAS-H SDD. IV: intravenous; PO: oral. N: Number of animals; Cₘₐₓ: Maximum observed plasma concentration; AUC: Area under the plasma concentration-time curve. ^{a} Median time (min-max); ^{b} Area under the plasma concentration-time curve from extrapolated 0 hour (0 ng/mL) to 24 hours for IV dose and from 0.25 to 24 hours for PO doses. ^{c} % F=Mean DN AUC (PO)/Mean DN AUC (IV), where DN: Dose-Normalized (for AUC or Cₘₐₓ).

FIG. 19. Sparsentan dose proportionality ratios.

FIG. 20. Mean plasma concentration of sparsentan over time for male rats administered one of six sparsentan formulations.

### DETAILED DESCRIPTION

The present disclosure relates to an amorphous form of a compound having the following structure (I):

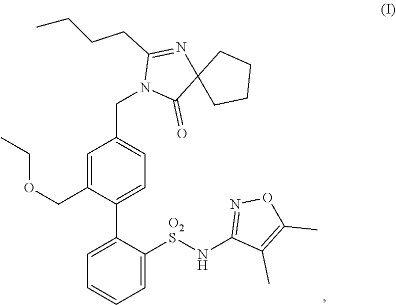 [SMILES: CCCCC1=NC2(CCCC2)C(=O)N1Cc1ccc(-c2ccccc2S(=O)(=O)Nc2noc(C)c2C)c(COCC)c1]

or a pharmaceutically acceptable salt thereof.

In the following description, certain specific details are set forth in order to provide a thorough understanding of various embodiments of the invention. However, one skilled in the art will understand that the invention may be practiced without these details.

Unless defined otherwise, all technical and scientific terms used herein have the same meaning as is commonly understood by one of skill in the art to which this invention belongs. As used herein, certain terms may have the following defined meanings.

Unless the context requires otherwise, throughout the present specification and claims, the word “comprise” and variations thereof, such as “comprises” and “comprising,” are to be construed in an open, inclusive sense, that is, as “including, but not limited to.”

As used in the specification and claims, “including” and variants thereof, such as “include” and “includes,” are to be construed in an open, inclusive sense; i.e., it is equivalent to “including, but not limited to.” As used herein, the terms “include” and “have” are used synonymously, which terms and variants thereof are intended to be construed as non-limiting.

As used in herein, the phrase “such as” refers to non-limiting examples.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment or to a single embodiment. Furthermore, the particular features, structures, or characteristics may be combined in any suitable manner in one or more embodiments.

As used in the specification and claims, the singular for “a,” “an,” and “the” include plural references unless the context clearly dictates otherwise. For example, the term “a cell” includes a plurality of cells, including mixtures thereof. Similarly, use of “a compound” for treatment of preparation of medicaments as described herein contemplates using one or more compounds of the invention for such treatment or preparation unless the context clearly dictates otherwise.

The use of the alternative (e.g., “or”) should be understood to mean either one, both, or any combination thereof of the alternatives.

“Optional” or “optionally” means that the subsequently described event or circumstance may or may not occur, and that the description includes instances where said event or circumstance occurs and instances in which it does not occur.

As used herein, “about” and “approximately” generally refer to an acceptable degree of error for the quantity measured, given the nature or precision of the measurements. Typical, exemplary degrees of error may be within 20%, 10%, or 5% of a given value or range of values. Alternatively, and particularly in biological systems, the terms “about” and “approximately” may mean values that are within an order of magnitude, potentially within 5-fold or 2-fold of a given value. When not explicitly stated, the terms “about” and “approximately” mean equal to a value, or within 20% of that value.

As used herein, numerical quantities are precise to the degree reflected in the number of significant figures reported. For example, a value of 0.1 is understood to mean from 0.05 to 0.14. As another example, the interval of values 0.1 to 0.2 includes the range from 0.05 to 0.24.

The compound having structure (I) forms salts that are also within the scope of this disclosure. Reference to a compound having structure (I) herein is understood to include reference to salts thereof, unless otherwise indicated. The term “salt(s),” as employed herein, denotes acidic or basic salts formed with inorganic or organic acids and bases. In addition, as the compound having structure (I) contains both a basic moiety and an acidic moiety, zwitterions (“inner salts”) may be formed and are included within the term “salt(s),” as used herein. Pharmaceutically acceptable (i.e., non-toxic, physiologically acceptable) salts are preferred, although other salts may be useful, e.g., in isolation or purification steps which may be employed during preparation. Salts of the compound having structure (I) may be formed, for example, by reacting the compound having structure (I) with an amount of acid or base, such as an equivalent amount, in a medium such as one in which the salt precipitates or in an aqueous medium followed by lyophilization.

The term “pharmaceutically acceptable salt” includes both acid and base addition salts.

Prodrugs and solvates of the compound having structure (I) are also contemplated. The term “prodrug” denotes a compound which, upon administration to a subject, undergoes chemical conversion by metabolic or chemical processes to yield a compound having structure (I), or a salt or solvate thereof. Solvates of the compound having structure (I) may be hydrates. Any tautomers are also contemplated.

Often crystallizations produce a solvate of the compound having structure (I), or a salt thereof. As used herein, the term “solvate” refers to an aggregate that comprises one or more molecules of a compound as disclosed herein with one or more molecules of solvent. In some embodiments, the solvent is water, in which case the solvate is a hydrate. Alternatively, in other embodiments, the solvent is an organic solvent. Thus, the compounds of the present disclosure may exist as a hydrate, including a monohydrate, dihydrate, hemihydrate, sesquihydrate, trihydrate, tetrahydrate, and the like, as well as the corresponding solvated forms. In some embodiments, the compounds disclosed herein may be a true solvate, while in other cases, the compounds disclosed herein merely retain adventitious water or are mixtures of water plus some adventitious solvent.

The invention disclosed herein is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the invention includes compounds produced by a process comprising administering a compound of this invention to a mammal for a period of time sufficient to yield a metabolic product thereof. Such products are typically identified by administering a radiolabeled compound of the invention in a detectable dose to an animal, such as rat, mouse, guinea pig, or monkey, or to a human, allowing sufficient time for metabolism to occur, and isolating its conversion products from the urine, blood, or other biological samples.

“Stable compound” and “stable structure” are meant to indicate a compound that is sufficiently robust to survive isolation to a useful degree of purity from a reaction mixture, and formulation into an efficacious therapeutic agent.

The term “subject” refers to a mammal, such as a domestic pet (for example, a dog or cat), or human. Preferably, the subject is a human.

The phrase “effective amount” refers to the amount which, when administered to a subject or patient for treating a disease, is sufficient to effect such treatment for the disease.

The term “dosage unit form” is the form of a pharmaceutical product, including, but not limited to, the form in which the pharmaceutical product is marketed for use. Examples include pills, tablets, capsules, and liquid solutions and suspensions.

“Treatment” or “treating” includes (1) inhibiting a disease in a subject or patient experiencing or displaying the pathology or symptomatology of the disease (e.g., arresting further development of the pathology or symptomatology); or (2) ameliorating a disease in a subject or patient that is experiencing or displaying the pathology or symptomatology of the disease (e.g., reversing the pathology or symptomatology); or (3) effecting any measurable decrease in a disease in a subject or patient that is experiencing or displaying the pathology or symptomatology of the disease.

Additional definitions are set forth throughout this disclosure.

### Amorphous Sparsentan

The instant disclosure provides an amorphous form of a compound having the following structure (I):

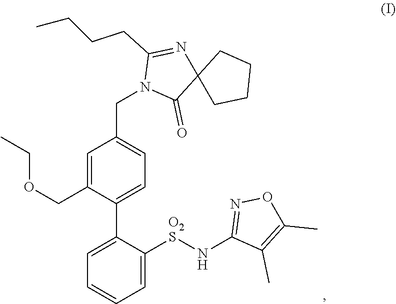 [SMILES: CCCCC1=NC2(CCCC2)C(=O)N1Cc1ccc(-c2ccccc2S(=O)(=O)Nc2noc(C)c2C)c(COCC)c1]

or a pharmaceutically acceptable salt thereof.

In a particular embodiment, the compound of structure I is sparsentan, or 2-[4-[(2-butyl-4-oxo-1,3-diazaspiro[4.4]non-1-en-3-yl)methyl]-2-(ethoxymethyl)phenyl]-N-(4,5-dimethyl-1,2-oxazol-3-yl)benzenesulfonamide. Sparsentan is a selective dual-acting receptor antagonist with affinity for endothelin (A type) receptors (“ETA” receptors) and angiotensin II receptors (Type 1) (“AT₁” receptors) (Kowala et al., JPET 309: 275-284, 2004). The compound of structure (I) may be prepared by methods such as those described in International Patent Application Publication No. WO2018/071784 A1, U.S. Patent Application Publication No. US 2015/0164865 A1, and U.S. Pat. No. 6,638,937 B2.

As used herein, “amorphous” refers to a substance whose constituent atoms, molecules, or ions are arranged randomly without a regular repeating pattern, as indicated by a lack of peaks when analyzed by powder X-ray diffraction (PXRD). Amorphous materials may have some localized crystallinity (i.e., regularity) but lack long-range order of the positions of the atoms. In contrast, “crystalline” refers to a material whose constituent atoms, molecules, or ions are arranged in an orderly repeating pattern.

In one embodiment, amorphous sparsentan provides greater bioavailability (e.g., higher Cₘₐₓ and AUC levels) compared to crystalline sparsentan when administered to a subject.

### Pharmaceutical Compositions

In one aspect, the present disclosure relates to pharmaceutical compositions comprising an amorphous form of a compound having structure (I) or a pharmaceutically acceptable salt thereof. The term “pharmaceutical composition” as used herein refers to a composition comprising an active ingredient with a pharmaceutically acceptable excipient. Pharmaceutical compositions may be used to facilitate administration of an active ingredient to an organism. Multiple techniques of administering a compound exist in the art, such as oral, injection, aerosol, parenteral, and topical administration. Pharmaceutical compositions can be obtained, for example, by reacting compounds with inorganic or organic acids such as hydrochloric acid, hydrobromic acid, sulfuric acid, nitric acid, phosphoric acid, methane sulfonic acid, ethanesulfonic acid, p-toluenesulfonic acid, salicylic acid, and the like.

As used herein, the term “physiologically acceptable excipient” refers to a physiologically and pharmaceutically suitable non-toxic and inactive material or ingredient that does not interfere with the activity of the active ingredient, including any adjuvant, carrier, glidant, sweetening agent, diluent, preservative, dye/colorant, flavor enhancer, surfactant, wetting agent, dispersing agent, suspending agent, stabilizer, isotonic agent, solvent, or emulsifier that has been approved by the United States Food and Drug Administration as being acceptable for use in humans or domestic animals.

In some embodiments, an excipient includes any substance, not itself a therapeutic agent, used as a carrier, diluent, adjuvant, or vehicle for delivery of a therapeutic agent to a subject or added to a pharmaceutical composition to improve its handling or storage properties or to permit or facilitate formation of a dose unit of the composition into a discrete article such as a capsule, tablet, film coated tablet, caplet, gel cap, pill, pellet, bead, and the like suitable for oral administration. For example, an excipient may be a surface active agent (or “surfactant”), carrier, diluent, disintegrant, binding agent, wetting agent, polymer, lubricant, glidant, coating or coating assistant, film forming substance, sweetener, solubilizing agent, smoothing agent, suspension agent, substance added to mask or counteract a disagreeable taste or odor, flavor, colorant, fragrance, or substance added to improve appearance of the composition, or a combination thereof.

Acceptable excipients include, for example, microcrystalline cellulose, lactose, sucrose, starch powder, maize starch or derivatives thereof, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, polyvinyl alcohol, saline, dextrose, mannitol, lactose monohydrate, lecithin, albumin, sodium glutamate, cysteine hydrochloride, croscarmellose sodium, sodium starch glycolate, hydroxypropyl cellulose, poloxamer (e.g., poloxamers 101, 105, 108, 122, 123, 124, 181, 182, 183, 184, 185, 188, 212, 215, 217, 231, 234, 235, 237, 238, 282, 284, 288, 331, 333, 334, 335, 338, 401, 402, 403, and 407, and poloxamer 105 benzoate, poloxamer 182 dibenzoate 407, and the like), sodium lauryl sulfate, colloidal silicon dioxide, and the like. Examples of suitable excipients for tablets and capsules include microcrystalline cellulose, silicified microcrystalline cellulose, lactose monohydrate, croscarmellose sodium, sodium starch, hydroxypropyl cellulose, poloxamer 188, sodium lauryl sulfate, colloidal silicon dioxide (colloidal silica), and magnesium stearate. Examples of suitable excipients for soft gelatin capsules include vegetable oils, waxes, fats, and semisolid and liquid polyols. Suitable excipients for the preparation of solutions and syrups include, for example, water, polyols, sucrose, invert sugar, and glucose. The compound can also be made in microencapsulated form. If desired, absorption enhancing preparations (for example, liposomes), can be utilized. Acceptable excipients for therapeutic use are well known in the pharmaceutical art, and are described, for example, in “Handbook of Pharmaceutical Excipients,” 5th edition (Raymond C Rowe, Paul J Sheskey and Sian C Owen, eds. 2005), and “Remington: The Science and Practice of Pharmacy,” 21st edition (Lippincott Williams & Wilkins, 2005).

In some embodiments, the above excipient can be present in an amount up to about 95% of the total composition weight, or up to about 85% of the total composition weight, or up to about 75% of the total composition weight, or up to about 65% of the total composition weight, or up to about 55% of the total composition weight, or up to about 45% of the total composition weight, or up to about 43% of the total composition weight, or up to about 40% of the total composition weight, or up to about 35% of the total composition weight, or up to about 30% of the total composition weight, or up to about 25% of the total composition weight, or up to about 20% of the total composition weight, or up to about 15% of the total composition weight, or up to about 10% of the total composition weight, or less.

As will be appreciated by those of skill in the art, the amounts of excipients will be determined by drug dosage and dosage form size. In some embodiments disclosed herein, the dosage form size is about 50 mg to 800 mg. In some embodiments disclosed herein, the dosage form size is about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, or about 800 mg. In another embodiment disclosed herein, the dosage form size is about 50 mg. In another embodiment disclosed herein, the dosage form size is about 100 mg. In another embodiment disclosed herein, the dosage form size is about 200 mg. In a further embodiment disclosed herein, the dosage form size is about 400 mg. In a further embodiment disclosed herein, the dosage form size is about 800 mg. In some embodiments disclosed herein, the dosage form size is 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, or 800 mg. In another embodiment disclosed herein, the dosage form size is 50 mg. In another embodiment disclosed herein, the dosage form size is 100 mg. In another embodiment disclosed herein, the dosage form size is 200 mg. In a further embodiment disclosed herein, the dosage form size is 400 mg. In a further embodiment disclosed herein, the dosage form size is 800 mg. One skilled in the art will realize that a range of weights may be made and are encompassed by this disclosure.

In one embodiment, the present disclosure provides a pharmaceutical composition comprising an amorphous form of a compound having structure (I) or a pharmaceutically acceptable salt thereof, wherein at least 50% of the compound by weight percent is present in an amorphous form. In one embodiment, the present disclosure provides a pharmaceutical composition comprising an amorphous form of a compound having structure (I) or a pharmaceutically acceptable salt thereof, wherein at least 60% of the compound by weight percent is present in an amorphous form. In one embodiment, the present disclosure provides a pharmaceutical composition comprising an amorphous form of a compound having structure (I) or a pharmaceutically acceptable salt thereof, wherein at least 70% of the compound by weight percent is present in an amorphous form. In one embodiment, the present disclosure provides a pharmaceutical composition comprising an amorphous form of a compound having structure (I) or a pharmaceutically acceptable salt thereof, wherein at least 80% of the compound by weight percent is present in an amorphous form. In one embodiment, the present disclosure provides a pharmaceutical composition comprising an amorphous form of a compound having structure (I) or a pharmaceutically acceptable salt thereof, wherein at least 90% of the compound by weight percent is present in an amorphous form. In one embodiment, the present disclosure provides a pharmaceutical composition comprising an amorphous form of a compound having structure (I) or a pharmaceutically acceptable salt thereof, wherein at least 95% of the compound by weight percent is present in an amorphous form. In one embodiment, the present disclosure provides a pharmaceutical composition comprising an amorphous form of a compound having structure (I) or a pharmaceutically acceptable salt thereof, wherein at least 98% of the compound by weight percent is present in an amorphous form. In one embodiment, the present disclosure provides a pharmaceutical composition comprising an amorphous form of a compound having structure (I) or a pharmaceutically acceptable salt thereof, wherein at least 99% of the compound by weight percent is present in an amorphous form.

In some embodiments, the pharmaceutical composition further comprises a pharmaceutically acceptable polymer. A “polymer” refers to a macromolecule comprised of one or more structural repeating units. Examples of polymers that can be used in the compositions disclosed herein include hydroxypropyl methylcellulose (hypromellose) (e.g., Methocel E3LV, Dow; Affinisol HPMC HME 15 cp, Dow), hypromellose acetate succinate LG (e.g., AQOAT-LG, Shin Etsu), hypromellose acetate succinate MG (e.g., AQOAT-MG, Shin Etsu); hypromellose acetate succinate HG (e.g., AQOAT-HG, Shin Etsu), hypromellose acetate succinate 716 (e.g., Affinisol HPMCAS 716, Dow), hypromellose acetate succinate 912 (e.g., Affinisol HPMCAS 912, Dow), hypromellose acetate succinate 126 (e.g., Affinisol HPMCAS 126, Dow), polyvinylpyrrolidone-vinyl acetate copolymer (e.g., Kollidon VA 64, BASF), polyvinyl caprolactam-polyvinyl acetate-polyethylene glycol graft copolymer (e.g., Soluplus®, BASF), polymethacrylate-based copolymers (e.g., EUDRAGIT® polymers including immediate release polymers, delayed release polymers (e.g., EUDRAGIT® L), and sustained release polymers (e.g., EUDRAGIT® RL and EUDRAGIT® RS)).

In one embodiment, the present disclosure provides a pharmaceutical composition comprising an amorphous form of a compound having structure (I) or a pharmaceutically acceptable salt thereof, and a polymer, wherein the weight ratio of the amorphous compound having structure (I), or pharmaceutically acceptable salt thereof, to the polymer is at least 95:5, 90:10, 85:15, 80:20, 75:25, 70:30, 65:35, 60:40, 55:45, 50:50, 45:55, 40:60, 35:65, 30:70, or 25:75. In one embodiment, the present disclosure provides a pharmaceutical composition comprising an amorphous form of a compound having structure (I) or a pharmaceutically acceptable salt thereof, and a polymer, wherein the weight ratio of the amorphous compound having structure (I), or pharmaceutically acceptable salt thereof, to the polymer is from 25:75 to 95:5.

In one aspect, the present disclosure relates to solid spray dried dispersion (“SDD”) formulations of amorphous sparsentan. Spray drying refers to the formation of solid particles by dispersing material within a liquid emulsion or slurry and evaporating the liquid by exposure to a hot gas. As disclosed herein, SDDs of amorphous sparsentan may be formed by spray drying an emulsion formed by dispersing sparsentan in a liquid medium, without or without the presence of a polymer. In one embodiment, the present disclosure provides an amorphous form of the compound of structure (I) or a pharmaceutically acceptable salt thereof, wherein the amorphous sparsentan or pharmaceutically acceptable salt thereof is produced by spray drying. In a further embodiment, the present disclosure provides a pharmaceutical composition comprising an amorphous form of the compound of structure (I) or pharmaceutically acceptable salt thereof and a polymer, wherein the amorphous compound and polymer are produced by spray drying.

### Formulations and Methods of Administration

In one aspect, the present disclosure relates to the formulation and administration of a pharmaceutical composition comprising an amorphous form of the compound of structure (I), or a pharmaceutically acceptable salt thereof, and pharmaceutically acceptable excipient. Techniques for formulation and administration of the compound of structure (I), or pharmaceutically acceptable salt thereof, may be found, for example, in “Remington's Pharmaceutical Sciences,” Mack Publishing Co., Easton, Pa., 18th edition, 1990. In some embodiments, the pharmaceutical composition is formulated as described below.

In some embodiments, surfactants are used. The use of surfactants as wetting agents in oral drug forms is described in the literature, for example in H. Sucker, P. Fuchs, P. Speiser, Pharmazeutische Technologie, 2nd edition, Thieme 1989, page 260. It is known from other papers, such as published in Advanced Drug Delivery Reviews (1997), 23, pages 163-183, that it is also possible to use surfactants, inter alia, to improve the permeation and bioavailability of pharmaceutical active compounds. Examples of surfactants include anionic surfactants, non-ionic surfactants, zwitterionic surfactants, and a mixture thereof. In some embodiments, the surfactant is selected from the group consisting of poly(oxyethylene) sorbitan fatty acid ester, poly(oxyethylene) stearate, poly(oxyethylene) alkyl ether, polyglycolated glyceride, poly(oxyethylene) castor oil, sorbitan fatty acid ester, poloxamer, fatty acid salt, bile salt, alkyl sulfate, lecithin, mixed micelle of bile salt and lecithin, glucose ester vitamin E TPGS (D-α-tocopheryl polyethylene glycol 1000 succinate), sodium lauryl sulfate (SLS), and the like, and mixtures thereof.

As used herein, the term “carrier” defines a chemical compound that facilitates the incorporation of a compound into cells or tissues. For example, dimethyl sulfoxide (DMSO) is a commonly utilized carrier, as it facilitates the uptake of many organic compounds into the cells or tissues of an organism. As used herein, the term “diluent” defines chemical compounds diluted in water that will dissolve the compound of interest as well as stabilize the biologically active form of the compound. Salts dissolved in buffered solutions are commonly utilized as diluents in the art. One commonly used buffered solution is phosphate buffered saline because it mimics the salt conditions of human blood. Because buffer salts can control the pH of a solution at low concentrations, a buffered diluent rarely modifies the biological activity of a compound. In some embodiments, a diluent selected from one or more of the compounds sucrose, fructose, glucose, galactose, lactose, maltose, invert sugar, calcium carbonate, lactose, starch, microcrystalline cellulose, lactose monohydrate, calcium hydrogen phosphate, anhydrous calcium hydrogen phosphate, a pharmaceutically acceptable polyol such as xylitol, sorbitol, maltitol, mannitol, isomalt, and glycerol, polydextrose, starch, and the like, or any mixture thereof, is used. Acceptable carriers or diluents for therapeutic use are well known in the pharmaceutical art, and are described, for example, in “Remington's Pharmaceutical Sciences,” 18th Ed., Mack Publishing Co., Easton, Pa. (1990).

In some embodiments, disintegrants such as starches, clays, celluloses, algins, gums, or crosslinked polymers are used, for example, to facilitate tablet disintegration after administration. Suitable disintegrants include, for example, crosslinked polyvinylpyrrolidone (PVP-XL), sodium starch glycolate, alginic acid, methacrylic acid DYB, microcrystalline cellulose, crospovidone, polacriline potassium, sodium starch glycolate, starch, pregelatinized starch, croscarmellose sodium, and the like. In some embodiments, the formulation can also contain minor amounts of nontoxic auxiliary substances such as wetting or emulsifying agents, pH buffering agents, and the like; for example, sodium acetate, sorbitan monolaurate, triethanolamine sodium acetate, triethanolamine oleate, sodium lauryl sulfate, dioctyl sodium sulfosuccinate, polyoxyethylene sorbitan fatty acid esters, and the like.

In some embodiments, binders are used, for example, to impart cohesive qualities to a formulation, and thus ensure that the resulting dosage form remains intact after compaction. Suitable binder materials include, but are not limited to, microcrystalline cellulose, gelatin, sugars (including, for example, sucrose, glucose, dextrose and maltodextrin), polyethylene glycol, waxes, natural and synthetic gums, polyvinylpyrrolidone, pregelatinized starch, povidone, cellulosic polymers (including, for example, hydroxypropyl cellulose (HPC), hydroxypropyl methylcellulose (HPMC), methyl cellulose, hydroxyethyl cellulose, and the like), and the like. Accordingly, in some embodiments, a formulations disclosed herein includes at least one binder to enhance the compressibility of the major excipient(s). For example, the formulation can include at least one of the following binders in the following ranges: from about 2% to about 6% w/w hydroxypropyl cellulose (Klucel); from about 2% to about 5% w/w polyvinylpyrrolidone (PVP); from about 1% to about 5% w/w methylcellulose; from about 2% to about 5% hydroxypropyl methylcellulose; from about 1% to about 5% w/w ethylcellulose; from about 1% to about 5% w/w sodium carboxy methylcellulose; and the like. One of ordinary skill in the art would recognize additional binders and/or amounts that can be used in the formulations described herein. As would be recognized by one of ordinary skill in the art, when incorporated into the formulations disclosed herein, the amounts of the major filler(s) and/or other excipients can be reduced accordingly to accommodate the amount of binder added in order to keep the overall unit weight of the dosage form unchanged. In one embodiment, a binder is sprayed on from solution, e.g., wet granulation, to increase binding activity.

In one embodiment, a lubricant is employed in the manufacture of certain dosage forms. For example, a lubricant may be employed when producing tablets. In one embodiment, a lubricant can be added just before the tableting step, and can be mixed with the other ingredients for a minimum period of time to obtain good dispersal. In some embodiments, one or more lubricants may be used. Examples of suitable lubricants include magnesium stearate, calcium stearate, zinc stearate, stearic acid, talc, glyceryl behenate, polyethylene glycol, polyethylene oxide polymers (for example, available under the registered trademarks of Carbowax® for polyethylene glycol and Polyox® for polyethylene oxide from Dow Chemical Company, Midland, Mich.), sodium lauryl sulfate, magnesium lauryl sulfate, sodium oleate, sodium stearyl fumarate, DL-leucine, colloidal silica, and others as known in the art. Typical lubricants are magnesium stearate, calcium stearate, zinc stearate, and mixtures of magnesium stearate with sodium lauryl sulfate. Lubricants may comprise from about 0.25% to about 50% of the tablet weight, typically from about 1% to about 40%, more typically from about 5% to about 30%, and most typically from 20% to 30%. In some embodiments, magnesium stearate can be added as a lubricant, for example, to improve powder flow, prevent the blend from adhering to tableting equipment and punch surfaces, and provide lubrication to allow tablets to be cleanly ejected from tablet dies. In some embodiments, magnesium stearate may be added to pharmaceutical formulations at concentrations ranging from about 0.1% to about 5.0% w/w, or from about 0.25% to about 4% w/w, or from about 0.5% w/w to about 3% w/w, or from about 0.75% to about 2% w/w, or from about 0.8% to about 1.5% w/w, or from about 0.85% to about 1.25% w/w, or from about 0.9% to about 1.20% w/w, or from about 0.85% to about 1.15% w/w, or from about 0.90% to about 1.1.% w/w, or from about 0.95% to about 1.05% w/w, or from about 0.95% to about 1% w/w. The above ranges are examples of typical ranges. One of ordinary skill in the art would recognize additional lubricants and/or amounts that can be used in the formulations described herein. As would be recognized by one of ordinary skill in the art, when incorporated into the pharmaceutical compositions disclosed herein, the amounts of the major filler(s) and/or other excipients may be reduced accordingly to accommodate the amount of lubricant(s) added in order to keep the overall unit weight of the dosage form unchanged.

In some embodiments, glidants are used. Examples of glidants include colloidal silicon dioxide, magnesium trisilicate, powdered cellulose, starch, talc, and calcium phosphate, and the like, and mixtures thereof.

In some embodiments, the formulations can include a coating, for example, a film coating. Where film coatings are included, coating preparations may include, for example, a film-forming polymer, a plasticizer, or the like. Also, the coatings may include pigments or opacifiers. Examples of film-forming polymers include hydroxypropyl methylcellulose, hydroxypropyl cellulose, methylcellulose, polyvinyl pyrrolidine, and starches. Examples of plasticizers include polyethylene glycol, tributyl citrate, dibutyl sebacate, castor oil, and acetylated monoglyceride. Furthermore, examples of pigments and opacifiers include iron oxides of various colors, lake dyes of many colors, titanium dioxide, and the like.

In some embodiments, color additives are included. The colorants can be used in amounts sufficient to distinguish dosage form strengths. In some embodiments, color additives approved for use in drugs (see 21 C.F.R. pt. 74) are added to the commercial formulations to differentiate tablet strengths. The use of other pharmaceutically acceptable colorants and combinations thereof is also encompassed by the current disclosure.

The pharmaceutical compositions as disclosed herein may include any other agents that provide improved transfer, delivery, tolerance, and the like. These compositions may include, for example, powders, pastes, jellies, waxes, oils, lipids, lipid (cationic or anionic) containing vesicles (such as Lipofectin®), DNA conjugates, anhydrous absorption pastes, oil-in-water and water-in-oil emulsions, emulsions of Carbowax (polyethylene glycols of various molecular weights), semi-solid gels, and semisolid mixtures containing Carbowax.

In various embodiments, alcohols, esters, sulfated aliphatic alcohols, and the like may be used as surface active agents; sucrose, glucose, lactose, starch, crystallized cellulose, mannitol, light anhydrous silicate, magnesium aluminate, magnesium metasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil, and the like may be used as smoothing agents; coconut oil, olive oil, sesame oil, peanut oil, and soya may be used as suspension agents or lubricants; cellulose acetate phthalate as a derivative of a carbohydrate such as cellulose or sugar, methyl acetatemethacrylate copolymer as a derivative of polyvinyl, or plasticizers such as ester phthalate may be used as suspension agents.

In one embodiment, a pharmaceutical composition as disclosed herein further comprises one or more of preservatives, stabilizers, dyes, sweeteners, fragrances, flavoring agents, and the like. For example, sodium benzoate, ascorbic acid, and esters of p-hydroxybenzoic acid may be included as preservatives. Antioxidants and suspending agents may also be included in the pharmaceutical composition.

In addition to being used as a monotherapy, the compounds and pharmaceutical compositions disclosed herein may also find use in combination therapies. Effective combination therapy may be achieved with a single pharmaceutical composition that includes multiple active ingredients, or with two or more distinct pharmaceutical compositions. Alternatively, each therapy may precede or follow the other by intervals ranging from minutes to months.

In some embodiments, one or more of, or any combination of, the listed excipients can be specifically included or excluded from the pharmaceutical compositions or methods disclosed herein.

Any of the foregoing formulations may be appropriate in treatments and therapies in accordance with the disclosure herein, provided that the one or more active ingredient in the pharmaceutical composition is not inactivated by the formulation and the formulation is physiologically compatible and tolerable with the route of administration (see also Baldrick P., “Pharmaceutical excipient development: the need for preclinical guidance.” Regul. Toxicol. Pharmacol. 32(2):210-8 (2000); Charman W. N., “Lipids, lipophilic drugs, and oral drug delivery-some emerging concepts.” J. Pharm. Sci. 89(8):967-78 (2000), and the citations therein for additional information related to formulations, excipients, and carriers well known to pharmaceutical chemists).

In some embodiments, the above excipients can be present in an amount up to about 95% of the total composition weight, or up to about 85% of the total composition weight, or up to about 75% of the total composition weight, or up to about 65% of the total composition weight, or up to about 55% of the total composition weight, or up to about 45% of the total composition weight, or up to about 43% of the total composition weight, or up to about 40% of the total composition weight, or up to about 35% of the total composition weight, or up to about 30% of the total composition weight, or up to about 25% of the total composition weight, or up to about 20% of the total composition weight, or up to about 15% of the total composition weight, or up to about 10% of the total composition weight, or less.

As will be appreciated by those of skill in the art, the amounts of excipients will be determined by drug dosage and dosage form size. In some embodiments disclosed herein, the dosage form size is about 50 mg to 800 mg. In another embodiment disclosed herein, the dosage form size is about 50 mg. In another embodiment disclosed herein, the dosage form size is about 100 mg. In another embodiment disclosed herein, the dosage form size is about 200 mg. In a further embodiment disclosed herein, the dosage form size is about 400 mg. In a further embodiment disclosed herein, the dosage form size is about 800 mg. One skilled in the art will realize that a range of weights may be made and are encompassed by this disclosure.

The pharmaceutical compositions of the present disclosure may be manufactured in a manner that is itself known, e.g., by means of conventional mixing, dissolving, granulating, dragee-making, levigating, emulsifying, encapsulating, entrapping, or tableting processes.

The pharmaceutical compositions of the present disclosure may provide low-dose formulations of the compound of structure (I), or a pharmaceutically acceptable salt thereof, in tablets, film coated tablets, capsules, caplets, pills, gel caps, pellets, beads, or dragee dosage forms. The formulations disclosed herein can provide favorable drug processing qualities, including, for example, rapid tablet press speeds, reduced compression force, reduced ejection forces, blend uniformity, content uniformity, uniform dispersal of color, accelerated disintegration time, rapid dissolution, low friability (preferable for downstream processing such as packaging, shipping, pick-and-pack, etc.) and dosage form physical characteristics (e.g., weight, hardness, thickness, friability) with little variation.

Proper formulation is dependent upon the route of administration chosen. Suitable routes for administering the compound of structure (I), or a pharmaceutically acceptable salt thereof, or a pharmaceutical composition comprising the same, may include, for example, oral, rectal, transmucosal, topical, or intestinal administration; and parenteral delivery, including intramuscular, subcutaneous, intravenous, intramedullary injections, intrathecal, direct intraventricular, intraperitoneal, intranasal, or intraocular injections. The compound of structure (I), or a pharmaceutically acceptable salt thereof, may also be administered in sustained or controlled release dosage forms, including depot injections, osmotic pumps, pills, transdermal (including electrotransport) patches, and the like, for prolonged or timed, pulsed administration at a predetermined rate.

Injectables can be prepared in conventional forms, either as liquid solutions or suspensions, solid forms suitable for solution or suspension in liquid prior to injection, or as emulsions. Suitable excipients may include, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include Hanks' solution, Ringer's solution, or physiological saline buffer. If desired, absorption enhancing preparations (for example, liposomes), may be utilized.

For transmucosal administration, penetrants appropriate to the barrier to be permeated may be used in the formulation.

Pharmaceutical formulations for parenteral administration, e.g., by bolus injection or continuous infusion, include aqueous solutions of the active compounds in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit, or almond oils, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, or dispersing agents. Alternatively, the active ingredient may be in powder form for constitution with a suitable vehicle, e.g., sterile pyrogen-free water, before use.

For oral administration, the compound of structure (I), or a pharmaceutically acceptable salt thereof, can be formulated by combining the active compound with pharmaceutically acceptable carriers known in the art. Such carriers enable the compound to be formulated as tablets, film coated tablets, pills, dragees, capsules, liquids, gels, get caps, pellets, beads, syrups, slurries, suspensions, and the like, for oral ingestion by a patient to be treated.

Pharmaceutical preparations for oral use can be obtained by combining the active compound with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; and cellulose preparations such as, for example, maize starch, wheat starch, rice starch, potato starch, gelatin, gum tragacanth, methyl cellulose, hydroxypropylmethyl-cellulose, sodium carboxymethylcellulose, or polyvinylpyrrolidone (PVP). If desired, disintegrating agents may be added, such as the cross-linked polyvinyl pyrrolidone, agar, or alginic acid or a salt thereof such as sodium alginate. Dragee cores having suitable coatings are also within the scope of the disclosure. For this purpose, concentrated sugar solutions may be used, which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, carbopol gel, polyethylene glycol, titanium dioxide, lacquer solutions, or suitable organic solvents or solvent mixtures. Dyestuffs or pigments may be added to the tablets or dragee coatings for identification or to characterize different combinations of active compound doses. In addition, stabilizers can be added. In some embodiments, formulations for oral administration are in dosages suitable for such administration. In some embodiments, formulations of the compound of structure (I), or a pharmaceutically acceptable salt thereof, have an acceptable immediate release dissolution profile and a robust, scalable method of manufacture.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, or lubricants such as talc or magnesium stearate, and, optionally, stabilizers. In soft capsules, the active compounds may be dissolved or suspended in suitable liquids, such as fatty oils, liquid paraffin, or liquid polyethylene glycols. In addition, stabilizers may be added.

For buccal administration, the compositions may take the form of tablets or lozenges formulated in a conventional manner.

For administration by inhalation, the compound of structure (I), or a pharmaceutically acceptable salt thereof, is conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Capsules and cartridges of, e.g., gelatin, for use in an inhaler or insufflator, may be formulated containing a powder mix of the compound and a suitable powder base such as lactose or starch.

Further disclosed herein are various pharmaceutical compositions well known in the pharmaceutical art for uses that include intraocular, intranasal, and intraauricular delivery. Suitable penetrants for these uses are generally known in the art. Pharmaceutical compositions for intraocular delivery include aqueous ophthalmic solutions of the active compounds in water-soluble form, such as eye drops, or in gellan gum (Shedden et al., Clin. Ther. 23(3):440-50, 2001) or hydrogels (Mayer et al., Ophthalmologica 210(2):101-3, 1996); ophthalmic ointments; ophthalmic suspensions, such as microparticulates, drug-containing small polymeric particles that are suspended in a liquid carrier medium (Joshi, J. Ocul. Pharmacol. 10(1):29-45, 1994), lipid-soluble formulations (Alm et al., Prog. Clin. Biol. Res. 312:447-58, 1989), and microspheres (Mordenti, Toxicol. Sci. 52(1):101-6, 1999); and ocular inserts. Such suitable pharmaceutical formulations may be formulated to be sterile, isotonic, and buffered for stability and comfort. Pharmaceutical compositions for intranasal delivery may also include drops and sprays often prepared to simulate in many respects nasal secretions, to ensure maintenance of normal ciliary action. As disclosed in “Remington's Pharmaceutical Sciences,” 18th Ed., Mack Publishing Co., Easton, Pa. (1990), and well known to those skilled in the art, suitable formulations are most often and preferably isotonic, slightly buffered to maintain a pH of 5.5 to 6.5, and most often and preferably include antimicrobial preservatives and appropriate drug stabilizers. Pharmaceutical formulations for intraauricular delivery include suspensions and ointments for topical application in the ear. Common solvents for such aural formulations include glycerin and water.

The compound of structure (I), or a pharmaceutically acceptable salt thereof, may also be formulated in rectal compositions such as suppositories or retention enemas, e.g., those containing conventional suppository bases such as cocoa butter or other glycerides.

In addition to the formulations described previously, the compound of structure (I), or pharmaceutically acceptable salt thereof, may also be formulated as a depot preparation. Such long acting formulations may be administered by implantation (for example subcutaneously or intramuscularly) or by intramuscular injection. Thus, for example, the compound of structure (I), or a pharmaceutically acceptable salt thereof, may be formulated with suitable polymeric or hydrophobic materials (for example as an emulsion in an acceptable oil) or ion exchange resins, or as sparingly soluble derivatives, for example, as a sparingly soluble salt.

For hydrophobic compounds, a suitable pharmaceutical carrier may be a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™ and 65% w/v polyethylene glycol 300, made up to volume in absolute ethanol. The proportions of a co-solvent system may be varied considerably without destroying its solubility and toxicity characteristics. Furthermore, the identity of the co-solvent components may be varied: for example, other low-toxicity nonpolar surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well-known examples of delivery vehicles or carriers for hydrophobic drugs. In some embodiments, certain organic solvents such as dimethylsulfoxide also may be employed.

Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Agents intended to be administered intracellularly may be administered using techniques well known to those of ordinary skill in the art. For example, such agents may be encapsulated into liposomes. Molecules present in an aqueous solution at the time of liposome formation are incorporated into the aqueous interior. The liposomal contents are both protected from the external micro-environment and, because liposomes fuse with cell membranes, are efficiently delivered into the cell cytoplasm. The liposome may be coated with a tissue-specific antibody. The liposomes will be targeted to and taken up selectively by the desired organ. Alternatively, small hydrophobic organic molecules may be directly administered intracellularly.

The compound of structure (I), or a pharmaceutically acceptable salt thereof, or pharmaceutical compositions comprising the same, may be administered to the patient by any suitable means. Examples of methods of administration include (a) administration though oral pathways, which includes administration in capsule, tablet, granule, spray, syrup, and other such forms; (b) administration through non-oral pathways such as rectal, vaginal, intraurethral, intraocular, intranasal, and intraauricular, which includes administration as an aqueous suspension, an oily preparation, or the like as a drip, spray, suppository, salve, ointment, or the like; (c) administration via injection, subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; and (e) administration topically; as deemed appropriate by those of skill in the art for bringing the compound of structure (I), or pharmaceutically acceptable salt thereof, into contact with living tissue.

Pharmaceutical compositions suitable for administration include compositions where the amorphous compound of structure (I), or a pharmaceutically acceptable salt thereof, is contained in an amount effective to achieve its intended purpose. The dose can be tailored to achieve a desired effect, but will depend on such factors as weight, diet, concurrent medication, and other factors that those skilled in the medical arts will recognize. More specifically, a therapeutically effective amount means an amount of compound effective to provide a therapeutic benefit to the subject being treated.

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will be dependent on many factors including the subject being treated, the severity of the affliction, the manner of administration, and the judgment of the prescribing physician. In one embodiment, the amorphous compound of structure (I), or pharmaceutically acceptable salt thereof, may be administered orally or via injection at a dose from 0.001 mg/kg to 2500 mg/kg of the patient's body weight per day. In a further embodiment, the dose range for adult humans is from 0.01 mg to 10 g/day. Tablets or other forms of presentation provided in discrete units may conveniently contain an amount of the compound of structure (I), or a pharmaceutically acceptable salt thereof, that is effective at such dosage or as a multiple of the same, for instance, units containing 5 mg to 1000 mg, usually from about 50 mg to about 800 mg. The dose employed will depend on a number of factors, including the age and sex of the patient, the precise disorder being treated, and its severity. Also, the route of administration may vary depending on the condition and its severity.

In cases wherein a salt is administered, dosages may be calculated as the dose of the free base.

In some embodiments, the dose range of the pharmaceutical composition administered to the patient can be from about 0.01 mg/kg to about 1000 mg/kg of the patient's body weight. The dosage may be a single one or a series of two or more given in the course of one or more days, as is needed by the patient.

In some embodiments, the daily dosage regimen for an adult human patient may be, for example, an oral dose of each active ingredient of between 0.1 mg and 2000 mg, or between 1 mg and 1500 mg, or between 5 mg to 1000 mg. In other embodiments, an oral dose of each active ingredient of between 1 mg and 1000 mg, between 50 mg and 900 mg, and between 50 mg to 800 mg is administered. In some embodiments, the oral dose is administered 1 to 4 times per day. In another embodiment, compositions of the amorphous compound of structure (I), or a pharmaceutically acceptable salt thereof, may be administered by continuous intravenous infusion, at a dose of each active ingredient up to 1000 mg per day. In some embodiments, the compound of structure (I), or a pharmaceutically acceptable salt thereof, will be administered for a period of continuous therapy, for example for a week or more, or for months or years.

In some embodiments, the dosing regimen of the amorphous compound of structure (I), or a pharmaceutically acceptable salt thereof, is administered for a period of time, which time period can be, for example, from at least about 4 weeks to at least about 8 weeks, from at least about 4 weeks to at least about 12 weeks, from at least about 4 weeks to at least about 16 weeks, or longer. The dosing regimen of the amorphous compound of structure (I), or pharmaceutically acceptable salt thereof, can be administered three times a day, twice a day, daily, every other day, three times a week, every other week, three times per month, once monthly, substantially continuously, or continuously.

In cases of local administration or selective uptake, the effective local concentration of the drug may not be related to plasma concentration. The amount of composition administered may be dependent on the subject being treated, on the subject's weight, the severity of the affliction, and the manner of administration.

In one embodiment, the present disclosure relates to a method of using an effective amount of the amorphous compound of structure (I) or pharmaceutically acceptable salt thereof in the treatment of a disease or disorder in a patient comprising administering to the patient a dosage of the amorphous compound of structure (I) or pharmaceutically acceptable salt thereof containing an amount of about 10 mg to about 1000 mg, of drug per dose, orally, at a frequency of three times per month, once monthly, once weekly, once every three days, once every two days, once per day, twice per day, three times per day, substantially continuously, or continuously, for the desired duration of treatment.

In another embodiment, the present disclosure provides a method of using an effective amount of the amorphous compound of structure (I) or pharmaceutically acceptable salt thereof in the treatment of a disease or disorder in a patient comprising administering to the patient a dosage containing an amount of about 50 mg to about 1000 mg, of drug per dose, orally, at a frequency of three times per month, once monthly, once weekly, once every three days, once every two days, once per day, twice per day, or three times per day, for the desired duration of treatment.

In yet another embodiment, the present disclosure provides a method of using an effective amount of the amorphous compound of structure (I) or pharmaceutically acceptable salt thereof in the treatment of a disease or disorder in a patient comprising administering to the patient a dosage containing an amount of about 50 mg of drug per dose, orally, at a frequency of three times per month, once monthly, once weekly, once every three days, once every two days, once per day, twice per day, or three times per day, for the desired duration of treatment.

In yet another embodiment, the present disclosure provides a method of using an effective amount of the amorphous compound of structure (I) or pharmaceutically acceptable salt thereof in the treatment of a disease or disorder in a patient comprising administering to the patient a dosage containing an amount of about 100 mg of drug per dose, orally, at a frequency of three times per month, once monthly, once weekly, once every three days, once every two days, once per day, twice per day, or three times per day, for the desired duration of treatment.

In yet another embodiment, the present disclosure provides a method of using an effective amount of the amorphous compound of structure (I) or pharmaceutically acceptable salt thereof in the treatment of a disease or disorder in a patient comprising administering to the patient a dosage containing an amount of about 200 mg of drug per dose, orally, at a frequency of three times per month, once monthly, once weekly, once every three days, once every two days, once per day, twice per day, or three times per day, for the desired duration of treatment.

In a further embodiment, the present disclosure provides a method of using an effective amount of the amorphous compound of structure (I) or pharmaceutically acceptable salt thereof in the treatment of a disease or disorder in a patient comprising administering to the patient a dosage containing an amount of about 400 mg of drug per dose, orally, at a frequency of three times per month, once monthly, once weekly, once every three days, once every two days, once per day, twice per day, or three times per day, for the desired duration of treatment.

In a further embodiment, the present disclosure provides a method of using an effective amount of the amorphous compound of structure (I) or pharmaceutically acceptable salt thereof in the treatment of a disease or disorder in a patient comprising administering to the patient a dosage containing an amount of about 800 mg of drug per dose, orally, at a frequency of three times per month, once monthly, once weekly, once every three days, once every two days, once per day, twice per day, or three times per day, for the desired duration of treatment.

In a further embodiments, the present disclosure provides a method of using an effective amount of the amorphous compound of structure (I) or pharmaceutically acceptable salt thereof in the treatment of a disease or disorder in a patient comprising administering to the patient a dosage from about 0.1 mg/kg to about 100 mg/kg, or from about 0.2 mg/kg to about 50 mg/kg, or from about 0.5 mg/kg to about 25 mg/kg of body weight (or from about 1 mg to about 2500 mg, or from about 50 mg to about 800 mg) of active compound per day, which may be administered in a single dose or in the form of individual divided doses, such as from 1 to 4 times per day.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in a form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising the compound of structure (I), or pharmaceutically acceptable salt thereof, formulated in a compatible pharmaceutical carrier may also be prepared, placed in an appropriate container, and labeled for treatment of an indicated condition.

### Uses and Methods of Treatment

Additionally, methods of treating diseases or disorders by administering a pharmaceutical composition comprising an amorphous form of a compound of structure (I), or pharmaceutically acceptable salt thereof, and a pharmaceutically acceptable excipient, are also within the scope of the present disclosure.

In one embodiment, the amorphous compound of structure (I) and pharmaceutically acceptable salts thereof are useful in the treatment of kidney diseases or disorders. Accordingly, in a specific embodiment, a method of treating kidney diseases or disorders is provided, comprising administering to a subject in need thereof a pharmaceutical composition comprising an effective amount of an amorphous compound of structure (I), or a pharmaceutically acceptable salt thereof.

In a further embodiment, the amorphous compound of structure (I) and pharmaceutically acceptable salts thereof, or a pharmaceutical composition comprising the amorphous compound of structure (I) or pharmaceutically acceptable salts thereof, are useful in the treatment of kidney diseases or disorders. In one embodiment, the amorphous compounds and pharmaceutical compositions disclosed herein are useful in the treatment of disorders related to renal, glomerular, and mesangial cell function, including acute (such as ischemic, nephrotoxic, or glomerulonephritis) and chronic (such as diabetic, hypertensive, or immune-mediated) renal failure, diabetic nephropathy, glomerular injury, renal damage secondary to old age or related to dialysis, nephrosclerosis (especially hypertensive nephrosclerosis), nephrotoxicity (including nephrotoxicity related to imaging and contrast agents and to cyclosporine), renal ischemia, primary vesicoureteral reflux, glomerulosclerosis, and the like. In one embodiment, the amorphous compounds and pharmaceutical compositions disclosed herein are useful in the treatment of disorders related to paracrine and endocrine function. In one embodiment, the amorphous compounds and pharmaceutical compositions disclosed herein are useful in the treatment of diabetic nephropathy, hypertension-induced nephropathy, and IGA-induced nephropathy.

In a still further embodiment, the amorphous compound of structure (I) and pharmaceutically acceptable salts thereof are useful in the reduction of general morbidity or mortality as a result of the above utilities.

In a further embodiment, the amorphous compound of structure (I) and pharmaceutically acceptable salts thereof, or a pharmaceutical composition comprising the amorphous compound of structure (I) or pharmaceutically acceptable salts thereof, are useful in the treatment of focal segmental glomerulosclerosis (FSGS). Accordingly, in a specific embodiment, a method of treating FSGS is provided, comprising administering to a subject in need thereof a pharmaceutical composition comprising an effective amount of an amorphous compound of structure (I), or a pharmaceutically acceptable salt thereof. In such embodiments, the FSGS may be primary, secondary, or genetic FSGS.

In a further embodiment, the amorphous compound of structure (I) and pharmaceutically acceptable salts thereof, or a pharmaceutical composition comprising the amorphous compound of structure (I) or pharmaceutically acceptable salts thereof, are useful in the treatment of IgA nephropathy. Accordingly, in a specific embodiment, a method of treating IgA nephropathy or hypertension-induced nephropathy is provided, comprising administering to a subject in need thereof a pharmaceutical composition comprising an effective amount of an amorphous compound of structure (I), or a pharmaceutically acceptable salt thereof.

In a further embodiment, the amorphous compound of structure (I) and pharmaceutically acceptable salts thereof, or a pharmaceutical composition comprising the amorphous compound of structure (I) or pharmaceutically acceptable salts thereof, are useful in the treatment of idiopathic membranous nephropathy (IMN). Accordingly, in a specific embodiment, a method of treating IMN is provided, comprising administering to a subject in need thereof a pharmaceutical composition comprising an effective amount of an amorphous compound of structure (I), or a pharmaceutically acceptable salt thereof.

In a further embodiment, the amorphous compound of structure (I) and pharmaceutically acceptable salts thereof, or a pharmaceutical composition comprising the amorphous compound of structure (I) or pharmaceutically acceptable salts thereof, are useful in the treatment of diabetic nephropathy and hypertension-induced nephropathy. Accordingly, in a specific embodiment, a method of treating diabetic nephropathy or hypertension-induced nephropathy is provided, comprising administering to a subject in need thereof a pharmaceutical composition comprising an effective amount of an amorphous compound of structure (I), or a pharmaceutically acceptable salt thereof.

In a further embodiment, the amorphous compound of structure (I) and pharmaceutically acceptable salts thereof, or a pharmaceutical composition comprising the amorphous compound of structure (I) or pharmaceutically acceptable salts thereof, are useful in the treatment of Alport syndrome. Accordingly, in a specific embodiment, a method of treating Alport syndrome is provided, comprising administering to a subject in need thereof a pharmaceutical composition comprising an effective amount of an amorphous compound of structure (I), or a pharmaceutically acceptable salt thereof. In a further embodiment, the amorphous compound of structure (I) and pharmaceutically acceptable salts thereof, or a pharmaceutical composition comprising the amorphous compound of structure (I) or pharmaceutically acceptable salts thereof, are useful in the treatment or prevention of hearing loss associated with Alport syndrome. In a specific embodiment, a method of treating or preventing hearing loss associated with Alport syndrome is provided, comprising administering to a subject in need thereof a pharmaceutical composition comprising an effective amount of an amorphous compound of structure (I), or a pharmaceutically acceptable salt thereof. As used herein, “prevention of, or preventing, hearing loss associated with Alport syndrome” refers to preventing the onset of, arresting hearing loss, or slowing the rate of hearing loss associated with Alport syndrome. For example, preventing hearing loss associated with Alport syndrome includes stabilizing hearing as well as slowing a decline in hearing.

In a further embodiment, the amorphous compound of structure (I) and pharmaceutically acceptable salts thereof, or a pharmaceutical composition comprising the amorphous compound of structure (I) or pharmaceutically acceptable salts thereof, are useful in the treatment of lupus nephritis. Accordingly, in a specific embodiment, a method of treating lupus nephritis is provided, comprising administering to a subject in need thereof a pharmaceutical composition comprising an effective amount of an amorphous compound of structure (I), or a pharmaceutically acceptable salt thereof.

In a further embodiment, the amorphous compound of structure (I) and pharmaceutically acceptable salts thereof, or a pharmaceutical composition comprising the amorphous compound of structure (I) or pharmaceutically acceptable salts thereof, are useful in the treatment of conditions associated with increased ET levels and/or increased angiotensin II levels and of endothelin-dependent or angiotensin II-dependent disorders. In a particular embodiment, the amorphous compound of structure (I) and pharmaceutically acceptable salts thereof, or a pharmaceutical composition comprising the amorphous compound of structure (I) or pharmaceutically acceptable salts thereof, are useful in the treatment of hypertension. By the administration of a composition having the amorphous compound, the blood pressure of a hypertensive mammalian (e.g., a human) host may be reduced. In one embodiment, the amorphous compound of structure (I) and pharmaceutically acceptable salts thereof, or a pharmaceutical composition comprising the amorphous compound of structure (I) or pharmaceutically acceptable salt thereof, are useful in the treatment of portal hypertension, hypertension secondary to treatment with erythropoietin, and low renin hypertension.

In one embodiment, any of the aforementioned uses or methods of treatment may comprise administering an amorphous form of the compound of structure (I), or pharmaceutically acceptable salt thereof, or pharmaceutical composition comprising the same, in combination with one or more other active ingredients, such as other therapeutic or diagnostic agents. For example, in one embodiment, one or more other therapeutic agents may be administered prior to, simultaneously with, or following the administration of the pharmaceutical composition comprising an effective amount of an amorphous form of a compound of structure (I), or a pharmaceutically acceptable salt thereof. If formulated as a fixed dose, such combination products may employ the compound of structure (I), or pharmaceutically acceptable salt thereof, within the dosage range described below, and the other active ingredient within its approved dosage range.

In one embodiment, the amorphous compound of structure (I), or pharmaceutically acceptable salt thereof, is used in conjunction with hemodialysis.

In any of the aforementioned embodiments, the amount of the amorphous compound having structure (I), or pharmaceutically acceptable salt thereof, administered to the subject may be from about 50 mg/day to about 1000 mg/day. For example, in one embodiment, the amount of the amorphous compound having structure (I), or pharmaceutically acceptable salt thereof, administered to the subject is from about 50 mg/day to about 800 mg/day. For example, in one embodiment, the amount of the amorphous compound having structure (I), or pharmaceutically acceptable salt thereof, administered to the subject is from about 200 mg/day to about 400 mg/day. In another embodiment, the amount of the amorphous compound having structure (I), or pharmaceutically acceptable salt thereof, administered to the subject is about 50 mg/day. In another embodiment, the amount of the amorphous compound having structure (I), or pharmaceutically acceptable salt thereof, administered to the subject is about 100 mg/day. In another embodiment, the amount of the amorphous compound having structure (I), or pharmaceutically acceptable salt thereof, administered to the subject is about 200 mg/day. In another embodiment, the amount of the amorphous compound having structure (I), or pharmaceutically acceptable salt thereof, administered to the subject is about 400 mg/day. In another embodiment, the amount of the amorphous compound having structure (I), or pharmaceutically acceptable salt thereof, administered to the subject is about 800 mg/day.

In any of the aforementioned embodiments, the amount of the amorphous compound having structure (I), or pharmaceutically acceptable salt thereof, administered to the subject may be from 50 mg/day to 1000 mg/day. For example, in one embodiment, the amount of the amorphous compound having structure (I), or pharmaceutically acceptable salt thereof, administered to the subject is from 50 mg/day to 800 mg/day. For example, in one embodiment, the amount of the amorphous compound having structure (I), or pharmaceutically acceptable salt thereof, administered to the subject is from 200 mg/day to 400 mg/day. In another embodiment, the amount of the amorphous compound having structure (I), or pharmaceutically acceptable salt thereof, administered to the subject is 50 mg/day. In another embodiment, the amount of the amorphous compound having structure (I), or pharmaceutically acceptable salt thereof, administered to the subject is 100 mg/day. In another embodiment, the amount of the amorphous compound having structure (I), or pharmaceutically acceptable salt thereof, administered to the subject is 200 mg/day. In another embodiment, the amount of the amorphous compound having structure (I), or pharmaceutically acceptable salt thereof, administered to the subject is 400 mg/day. In another embodiment, the amount of the amorphous compound having structure (I), or pharmaceutically acceptable salt thereof, administered to the subject is 800 mg/day.

In one embodiment, the dosing regimen comprises administering the amorphous compound having structure (I) in an amount of 50 mg/day. In one embodiment, the dosing regimen comprises administering the amorphous compound having structure (I) in an amount of 100 mg/day. In one embodiment, the dosing regimen comprises administering the amorphous compound having structure (I) in an amount of 200 mg/day. In one embodiment, the dosing regimen comprises administering the amorphous compound having structure (I) in an amount of 400 mg/day. In one embodiment, the dosing regimen comprises administering the amorphous compound having structure (I) in an amount of 800 mg/day. In another embodiment, the dosing regimen comprises administering the amorphous compound having structure (I) in an amount of 50 mg/day for 8 weeks, 26 weeks, or 8 months. In another embodiment, the dosing regimen comprises administering the amorphous compound having structure (I) in an amount of 100 mg/day for 8 weeks, 26 weeks, or 8 months. In another embodiment, the dosing regimen comprises administering the amorphous compound having structure (I) in an amount of 200 mg/day for 8 weeks, 26 weeks, or 8 months. In another embodiment, the dosing regimen comprises administering the amorphous compound having structure (I) in an amount of 400 mg/day for 8 weeks, 26 weeks, or 8 months. In another embodiment, the dosing regimen comprises administering the amorphous compound having structure (I) in an amount of 800 mg/day for 8 weeks, 26 weeks, or 8 months.

In any of the aforementioned embodiments, the amorphous compound may be a compound having structure (I).

In any of the aforementioned embodiments, the method may further comprise administering to said subject one or more additional therapeutic agents.

In any of the aforementioned embodiments, the subject may be an adult or may be 18 years old or younger. In some embodiments, the subject is 18 years old or younger.

In some embodiments, the present disclosure provides a pharmaceutical composition comprising an amorphous compound having structure (I), or a pharmaceutically acceptable salt thereof, and a pharmaceutically acceptable excipient for use in the aforementioned methods.

In some embodiments, the present disclosure provides for the use of the aforementioned compounds or pharmaceutical compositions in the manufacture of a medicament for use in the therapeutic methods described herein. In some embodiments, the present disclosure provides for the use of a pharmaceutical composition comprising an amorphous compound having structure (I), or a pharmaceutically acceptable salt thereof, in the manufacture of a medicament for use in the aforementioned therapeutic methods.

### EXAMPLES

### Example 1

### Amorphous Sparsentan

Amorphous sparsentan was prepared by spray drying a mixture of crystalline sparsentan and acetone, using a Büchi B-290 with a 2-fluid nozzle, 1.5 mm Air Cap, and 0.7 mm Liquid tip, at the settings shown in Table 1.

TABLE 1
Spray drying process parameters for preparing
amorphous sparsentan.

| Parameter | |
|---|---|
| Batch size, total solids | 5 g |
| Lot number | R6-678-70 |
| Date of manufacture | 14 Nov. 2018 |
| Spray solvent | Acetone |
| Spray solution | 10% |
| composition (wt % total | |
| solids) | |
| Drying gas mode | Recycle |
| Cyclone used | High Efficiency |
| Solution flow rate | 20 mL/min (setting) |
| | 12-14 g/min (actual) |
| Atomization pressure | 26 psi |
| Inlet temperature | 68-70° C. |
| Outlet temperature | 37-38° C. |
| Condenser temperature | −20° C. |
| Secondary drying | 24 h/ |
| time/temperature | 30-35° C. |
| SDD yield | 44.3% |
| Residual acetone (ppm) | Not detected |

The resulting spray dried material was characterized using powder X-ray diffraction (PXRD) analysis and modulated differential scanning calorimetry (MDSC). PXRD was obtained using a Rigaku Miniflex 6G at the following measurement conditions: Radiation Source—Cu-Kα (1.5406 Å); Scan Mode—Coupled 2θ/θ; Scan Range—5°-40°; Scan Speed—0.9°/min; Step Increment-0.005°; Voltage—40 kV; Current-15 mA; Rotation—30 rpm; Divergence Slit-0.625 mm. MDSC was performed using a TA Discovery DSC2500 with RCS90 chiller at the following conditions: Scan Mode—Modulated; Temperature Range—0° C.-200° C.; Heating Rate—2.0° C./min; Mod. Period—60 s; Mod. Amplitude—±1.0° C.; Pan/Lid type: Non-Hermetic; Replicates—n=3.

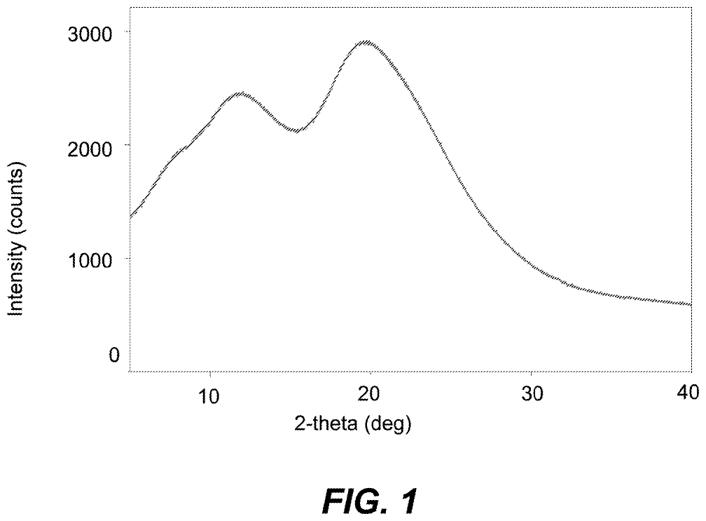

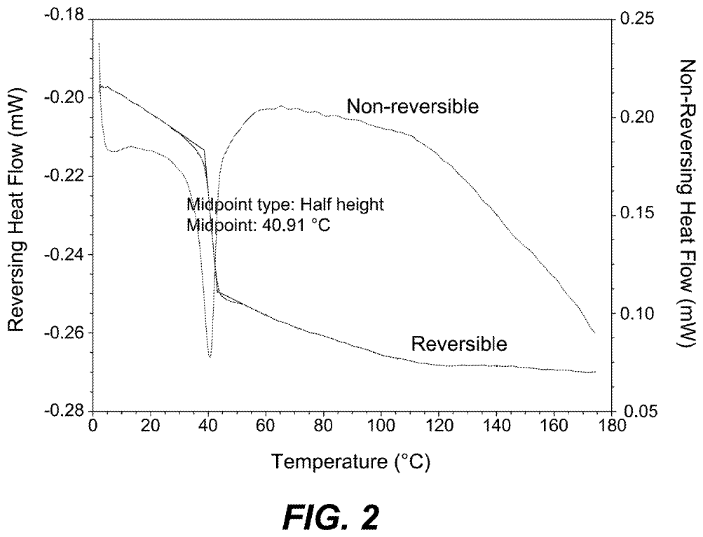

The spray dried sparsentan was amorphous by PXRD (FIG. 1) and MDSC (FIG. 2). MDSC showed a single glass transition temperature (Tg), indicating good homogeneity (FIG. 2).

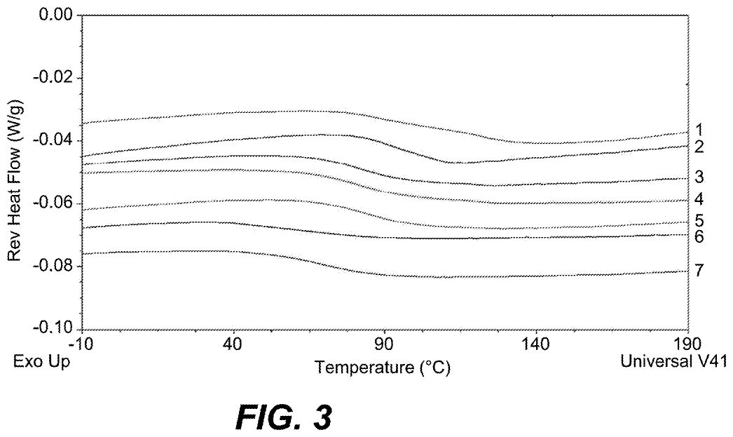

A mortar and pestle was used to generate physical mixtures of sparsentan with various polymers at a 20:80 sparsentan:polymer weight ratio. Resulting mixtures were heated in DSC pans to past the melting point of sparsentan (147° C.), held isothermally for 10 mins to dissolve the sparsentan into the polymer matrix, and then quickly quenched to −25° C. to trap the material in an amorphous state. Samples were then heated up to 200° C. utilizing modulation and the instrument parameters in Table 2 and characterized by MDSC. All mixtures showed a single Tg, indicating a homogenous material, and no melting event was observed during the ramp up to 200° C., indicating than an amorphous material was generated and was thermodynamically stable. Overlaid thermograms are shown in FIG. 3, with observed Tg values shown in Table 3.

TABLE 2
MDSC parameters used for sparsentan:polymer physical mixtures.

| Parameter | Value |
|---|---|
| Instrument | TA Q200, RCS 90 |
| Sample pans | Al, Non-hermetic |
| Temperature range | 25-200° C. |
| Heating rate | 2.0° C./min |
| Scanning mode | Modulated |
| Modulation frequency | 60s |
| Modulation amplitude | 1° C. |

TABLE 3
MDSC data for 20:80 sparsentan:polymer physical mixtures.

| Formulation | Tg, ° C. (avg.) |
|---|---|
| 20:80 Sparsentan:Eudragit | 103.8 ± 1.9 |
| L100-55 Physical Mixture | |
| 20:80 Sparsentan:PVP-VA | 94.0 ± 0.4 |
| Physical Mixture | |
| 20:80 Sparsentan:Affinisol 716 | 82.5 ± 1.9 |
| Physical Mixture | |
| 20:80 Sparsentan:Affinisol 912 | 80.3 ± 2.6 |
| Physical Mixture | |
| 20:80 Sparsentan:Affinisol 126 | 84.2 ± 1.2 |
| Physical Mixture | |
| 20:80 Sparsentan:HPMC | 61.3 ± 1.6 |
| HME Grade Physical Mixture | |
| 20:80 Sparsentan:Soluplus | 67.2 ± 1.7 |
| Physical Mixture | |

### Example 2

### Spray Dried Dispersions of Sparsentan and Polymer

Amorphous solid spray dried dispersion (“SDD”) formulations of sparsentan were manufactured by spray drying sparsentan with a polymer.

Sparsentan was spray dried with one of the polymers shown in Table 4. For each of the polymers, mixtures of sparsentan to polymer at 25:75 and 50:50 weight ratios were used. These mixtures were spray dried from 100% acetone, with 80:20 MeOH:H₂O used for HPMC E3LV SDD formulations. The spray drying parameters are shown in Table 5.

TABLE 4
Polymers used in spray dried dispersions of Example 2.

| Material | Trade name | Abbreviation | Manufacturer |
|---|---|---|---|
| Hydroxypropyl | Methocel E3LV | HPMC E3LV | Dow |
| methylcellulose | | | |
| (Hypromellose) | | | |
| Hypromellose | AQOAT-HG | HPMCAS-H | Shin Etsu |
| acetate succinate | | | |
| HG | | | |
| Polyvinylpyrrolidone- | Kollidon VA 64 | PVP-VA | BASF |
| Vinyl acetate | | | |
| copolymer | | | |
| Polyvinyl | Soluplus ® | Soluplus ® | BASF |
| caprolactam- | | | |
| polyvinyl acetate- | | | |
| polyethylene glycol | | | |
| graft copolymer | | | |

TABLE 5
Spray drying parameters used in Example 2.

| Parameter | Value |
|---|---|
| Spray dryer | Büchi B290 |
| Cyclone | High Efficiency |
| Drying gas mode | Recycle |
| Condenser temperature | -20° C. |
| Solvent | Acetone for PVP-VA, |
| | HPMCAS-H and Soluplus |
| | formulations, 80:20 |
| | MeOH:H₂O for HPMC |
| | Formulations |
| Batch size, total solids | 8-10 g |
| Solution composition | 10% solids, 8% for HPMC |
| | Formulations |
| Atomization pressure | 26 psi |
| Solution feed rate | 17-20 g/min for acetone |
| | solutions |
| | 13-15 g/min for MeOH:H₂O |
| | solutions |
| Inlet temperature | 83-87° C. (Acetone) |
| | 146-156° C. (80:20 |
| | MeOH:H₂O) |
| Outlet temperature | 43-46° C. (Acetone) |
| | 56-59° C. (80:20 MeOH:H₂O) |
| Secondary drying | ca. 24 hr at 40° C. in a |
| | forced-air convection oven |

A secondary tray drying process was used to remove residual solvent after the initial spray drying process. In this operation, the “wet” SDD was heated to 40° C. and stored in a convection tray oven for 24 hours. The residual solvent content of the SDDs was measured by GC headspace analysis (GC-HS) after secondary drying. Measurements were made using an HP 6890 series GC equipped with an Agilent 7697A headspace sampler. A 30 m×0.32 mm×1.8 μcapillary column with 6% cyanopropylphenyl 94% dimethylpolysiloxane GC column was used for the testing. GC samples were prepared by dissolving ˜100 mg sample in 4 mL dimethyl sulfoxide (DMSO). The GC method parameters are summarized in Table 6.

TABLE 6
Headspace GC method parameters.

| Parameter | Value |
|---|---|
| Sample temperature | 105° C. |
| Sample loop | 110° C. |
| temperature | |
| Transfer line | 115° C. |
| temperature | |
| GC cycle time | 45 min |
| Vial equilibration time | 30 min |
| Injection time | 1.00 min |
| Injection loop size | 1 mL |
| Post injection purge | 100 mL/min; 1 min |
| Carrier gas | N₂, ≥99.999% |
| Carrier gas flow | 25 mL/min |
| Vial pressure | 15.0 psi |

The residual solvent in all formulations was well below the acetone (5000 ppm) and MeOH (3000 ppm) limit set forth by the International Conference on Harmonization (ICH).

### Analytical Methods

All spray dried dispersions were characterized using MDSC, PXRD, and scanning electron microscopy (SEM).

MDSC was performed using a TA Instruments Q200 differential scanning calorimeter equipped with a TA instruments Refrigerated Cooling System 90. MDSC was used to measure glass transition temperature (Tg), cold crystallization (Tc), defined as a crystallization event at a temperature lower than the melt temperature, and melting temperature (Tm). Samples were placed in non-hermetic aluminum pans and heated at a constant rate of 2.0° C./min over a 25-200° C. temperature range. The system was purged by nitrogen flow at 50 mL/min to ensure inert atmosphere through the course of measurement. A summary of MDSC analysis parameters is shown in Table 7.

TABLE 7
MDSC parameters used in Example 2.

| Parameter | Value |
|---|---|
| Instrument | TA Q200, RCS 90 |
| Sample pans | Al, Non-hermetic |
| Temperature range | 25-200° C. |
| Heating rate | 2.0° C./min |
| Scanning mode | Modulated |
| Modulation frequency | 60s |
| Modulation amplitude | 1° C. |

PXRD was performed using a Rigaku MiniFlex 6G X-ray diffractometer to evaluate the crystallinity of spray dried materials. Amorphous materials give an “amorphous halo” diffraction pattern, absent of discrete peaks that would be found in a crystalline material. The samples were irradiated with monochromatized Cu Kα radiation and analyzed between 5° and 40° with a continuous scanning mode. Samples were rotated at 30 rpm during analysis to minimize preferred orientation effects. A summary of PXRD analysis parameters is shown in Table 8.

TABLE 8
PXRD parameters used in Example 2.

| Parameter | Value |
|---|---|
| Instrument | Rigaku Miniflex 6G |
| Radiation source | Cu-Kα (1.5406 Å), Line Focus |
| | 0.4 mm × 12 mm |
| Scan type | Coupled 2θ/θ |
| Scan range | 5°-40° |
| Step increment | 0.005° |
| Ramp rate | 0.9°/min |
| Voltage | 40 kV |
| Current | 15 mA |
| Rotation | 30 r/min |
| Holder | Zero-Background Cup |
| Slit width | 1.0 mm |
| Knife-edge width | 1.0 mm |

SEM samples were prepared by dispersing powder onto an adhesive carbon-coated sample stub a coating with a thin conductive layer of gold using a Polaron Autocoater E5200. Samples were analyzed using a FEI Quanta 200 SEM fitted with an Everhart-Thornley (secondary electron) detector, operating in high vacuum mode. Micrographs at various magnifications were captured for qualitative particle morphology analysis. Experimental parameters including spot size, working distance, and acceleration voltage were varied from sample to sample to obtain the best imaging conditions, and are documented in the caption of each SEM micrograph.

### Results

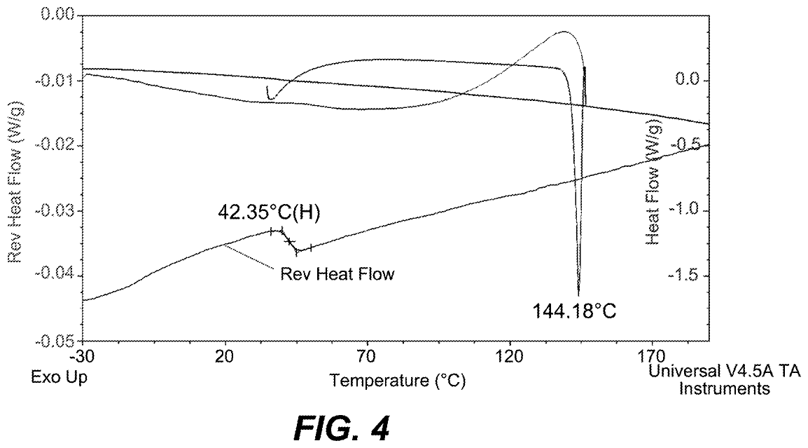

Thermal properties, melting temperature (Tm), glass transition temperature (Tg), and crystallization temperature (Tc) of sparsentan was measured by MDSC. The Tg was measured via a melt-quench technique, heating past its melting temperature and rapidly cooling to trap the molten material in an amorphous state. The resulting sample was analyzed by MDSC and a Tg of 42° C. was determined (FIG. 4).

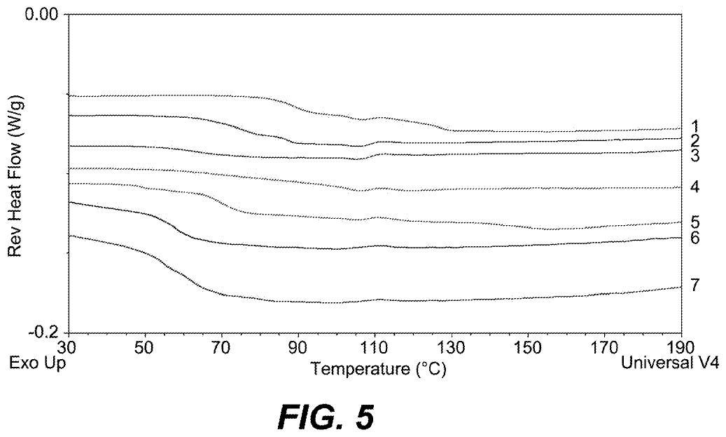

Thermal analysis done by MDSC revealed multiple SDD dispersions having a single yet broad Tg (FIG. 5) indicating a non-homogenous amorphous solid dispersion with poor homogeneity (Table 9). Relatively high glass transition temperatures were observed for most formulations, indicating good physical stability (i.e., the propensity of the sparsentan to recrystallize during long-term storage is low). SDDs stored below the Tg at a given condition should exhibit low mobility of the drug in the glass dispersion.

TABLE 9
Glass transition temperatures for spray dried sparsentan
dispersions as determined by MDSC.

| | | Avg. Measured |
|---|---|---|
| Formulation | Lot # | Tg (° C.) |
| 25:75 Sp:PVP-VA | R6-678-1 | 87.6 |
| SDD | | |
| 25:75 Sp:HPMCAS-H | R6-678-2 | 77.5 |
| SDD | | |
| 25:75 Sp:Soluplus | R6-678-3 | 63.6 |
| SDD | | |
| 25:75 Sp:HPMC | R6-678-4 | 78.6 |
| E3LV SDD | | |
| 50:50 Sp:PVP-VA | R6-678-5 | 70.4 |
| SDD | | |
| 50:50 Sp:HPMCAS-H | R6-678-6 | 58.5 |
| SDD | | |
| 50:50 Sp:Soluplus | R6-678-7 | 56.6 |
| SDD | | |
| 50:50 Sp:HPMC | R6-678-8 | 62.8 |
| E3LV SDD | | |

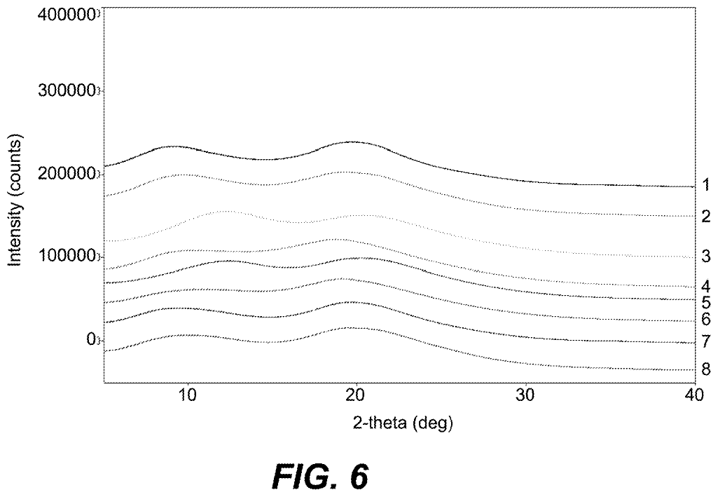

PXRD analysis showed that the SDDs were amorphous dispersions and no crystalline peaks were observed in the SDD diffractograms (FIG. 6).

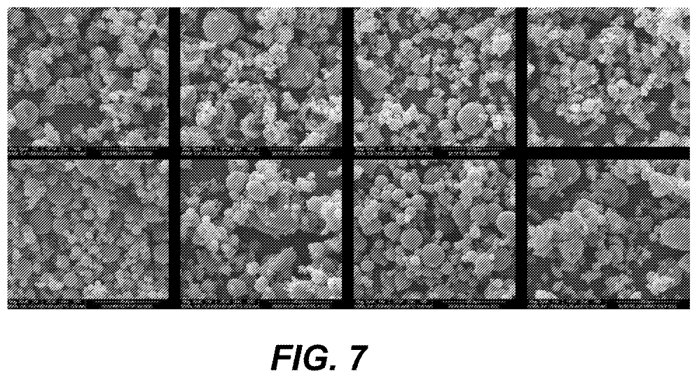

Surface morphology of the SDD particles was characterized using scanning electron microscopy. The SEM images in FIG. 7 show images of the sparsentan SDDs at 5000× magnification. Typical SDD morphology was observed consisting of whole and collapsed spheres with smooth surfaces. No crystalline material was observed in any samples.

Sparsentan is stable as a neat amorphous form, with no crystallization or melting events observed in a modulated ramp up to 200° C. MDSC experiments on sparsentan SDDs revealed nonhomogeneous dispersions with broad glass transition temperatures.

### Example 3

### Amorphous Spray Dried Dispersions of Sparsentan with High Drug Loading

Amorphous solid dispersion formulations of sparsentan having higher drug loading amounts were manufactured by spray drying a mixture of crystalline sparsentan alone and acetone; crystalline sparsentan and polyvinylpyrrolidone-vinyl acetate copolymer (Kollidon VA 64, BASF; “PVP-VA”) present in acetone at a 80:20 ratio; or crystalline sparsentan and PVP-VA present in acetone at a 65:35 ratio, using a Büchi B-290 with a 2-fluid nozzle, 1.5 mm Air Cap, and 0.7 mm Liquid tip, at the settings shown in Table 10 and according to the same general methodology as described in Example 2. The resulting spray dried material was characterized using PXRD analysis and MDSC as described in Example 1.

TABLE 10
Spray drying process parameters for preparing amorphous
sparsentan formulations of Example 3.

| | | 80:20 | 65:35 |
|---|---|---|---|
| | 100% | Sparsentan: | Sparsentan: |
| Parameter | Sparsentan | PVP-VA | PVP-VA |
| Batch size, total solids | 5 g | 6.25 g (5 g | 7.7 g (5 g |
| | | sparsentan) | sparsentan) |
| Lot number | R6-678-70 | R6-678-72 | R6-678-71 |
| Date of manufacture | 14 Nov. 2018 | 15 Nov. 2018 | 15 Nov. 2018 |
| Spray solvent | Acetone | Acetone | Acetone |
| Spray solution | 10% | 10% | 10% |
| composition (wt % | | | |
| total solids) | | | |
| Drying gas mode | Recycle | Recycle | Recycle |
| Cyclone used | High | High | High |
| | Efficiency | Efficiency | Efficiency |
| Solution flow rate | 20 mL/min | 20 mL/min | 20 mL/min |
| | (setting) | (setting) | (setting) |
| | 12-14 g/min | 17-23 g/min | 17-22 g/min |
| | (actual) | (actual) | (actual) |
| Atomization pressure | 26 psi | 26 psi | 26 psi |
| Inlet temperature | 68-70° C. | 79-81° C | 81-83° C. |
| Outlet temperature | 37-38° C. | 41-43° C | 41-43° C. |
| Condenser | −20° C. | −20° C. | −20° C. |
| temperature | | | |
| Secondary drying | 24 h/ | 24 h/ | 24 h/ |
| time/temperature | 30-35° C. | 30-35° C. | 30-35° C. |
| SDD yield | 44.3% | 76.4% | 78.4% |
| Residual acetone | Not detected | Not detected | Not detected |
| (ppm) | | | |

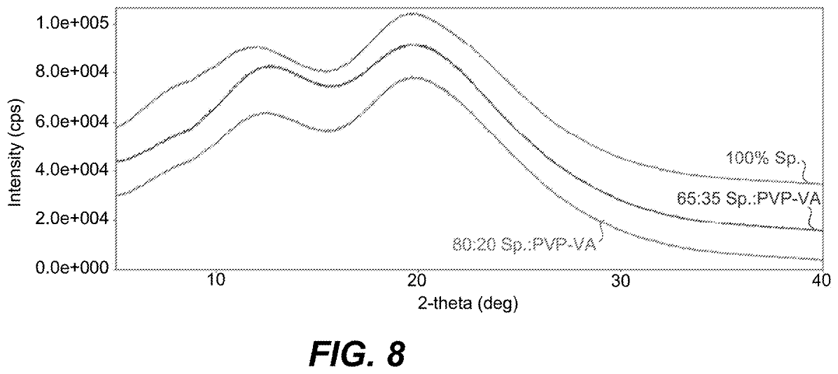

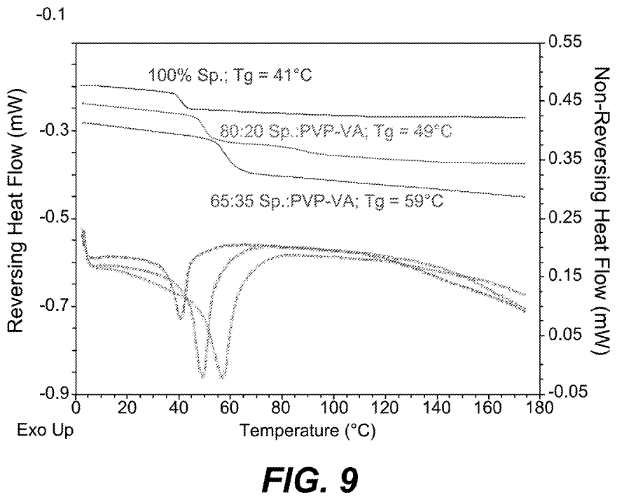

All three spray dried dispersions (“SDDs”) were amorphous by PXRD (FIG. 8) and MDSC (FIG. 9).

### Example 4

### Amorphous Sparsentan Formulations Provide Greater Oral Bioavailability

The pharmacokinetics of crystalline and amorphous forms of sparsentan were investigated.

Sparsentan was administered intravenously as a bolus injection (“IV”) or orally (“PO”) to male Sprague Dawley rats in a single dose, as described in Table 11. Four sparsentan formulations were tested: crystalline sparsentan (“Crystalline Sp”); spray dried dispersion particles formed from a 50:50 mixture of sparsentan and polyvinylpyrrolidone-vinyl acetate copolymer (“50:50 Sp:PVP-VA SDD”); spray dried dispersion particles formed from a 50:50 mixture of sparsentan and hydroxypropyl methylcellulose (“50:50 Sp:HPMC E3LV SDD”); and spray dried dispersion particles formed from a 50:50 mixture of sparsentan and hypromellose acetate succinate HG (“50:50 Sp: HPMCAS-H SDD”). Formulations were administered in a vehicle comprised of either a mixture of PEG400:ethanol:sterile water (30:30:40 v/v/v) (“A”) or a mixture of 0.5% Methocel A4M, 0.1% Tween-80 in purified water (“B”).

TABLE 11
Treatment groups and dose levels.

| | | | | | Target | Target | Target dose |
|---|---|---|---|---|---|---|---|
| | | | | | dose level | dose conc. | volume |
| Group | n | Formulation | Vehicle | Dose route | (mg/kg) | (mg/mL)¹ | (mL/kg) |
| 1 | 3 | Crystalline Sp | A | IV | 1 | 2 | 0.5 |
| 2 | 3 | Crystalline Sp | B | PO | 20 | 2 | 10 |
| 3 | 3 | Crystalline Sp | B | PO | 60 | 6 | 10 |
| 4 | 3 | 50:50 | B | PO | 20 | 2 | 10 |
| | | Sp:PVP-VA SDD | | | | | |
| 5 | 3 | 50:50 | B | PO | 60 | 6 | 10 |
| | | Sp:PVP-VA SDD | | | | | |
| 6 | 3 | 50:50 | B | PO | 20 | 2 | 10 |
| | | Sp:HPMC E3LV SDD | | | | | |
| 7 | 3 | 50:50 | B | PO | 60 | 6 | 10 |
| | | Sp:HPMC E3LV SDD | | | | | |
| 8 | 3 | 50:50 | B | PO | 20 | 2 | 10 |
| | | Sp:HPMCAS-H SDD | | | | | |
| 9 | 3 | 50:50 | B | PO | 60 | 6 | 10 |
| | | Sp:HPMCAS-H SDD | | | | | |

A: PEG400:ethanol:sterile water (30:30:40 v/v/v)
B: 0.5% Methocel A4M, 0.1% Tween-80 in purified water
IV: Intravenous, given as bolus injection, via a tail vein; given fed
PO: Oral, via a gavage needle; given fasted
¹Target dose concentration (mg/mL).

Blood samples were collected at 0.083 (Group 1 only), 0.25, 0.5, 1, 2, 4, 6, 8, and 24 hours post dose. Blood was collected into tubes containing K2EDTA and centrifuged, and resulting plasma samples were obtained. For each group, the following PK parameters were determined: maximum observed plasma concentration (Cₘₐₓ), time of maximum observed plasma concentration (Tₘₐₓ), and area under the plasma concentration-time curve (AUC). AUC from time 0 to 24 hours (AUC_{0-24 hr}) was calculated for all groups with at least three consecutive quantifiable concentrations. For the IV dose, extrapolated null concentration (C₀) was calculated and used as Cₘₐₓ at time zero. For the oral doses, extrapolated value at time zero was assigned as 0.0 ng/m L. The AUC based on actual collection time point was from 0.083 to 24 hours for IV group and from 0.25 to 24 hour for PO group. Absolute bioavailability evaluation was determined as “% F” between single dose IV and oral dose, calculated as follows:

% F=Mean Dose Normalized AUC_{0-24 hr}(PO)/Mean Dose Normalized AUC_{0-24 hr} (IV).

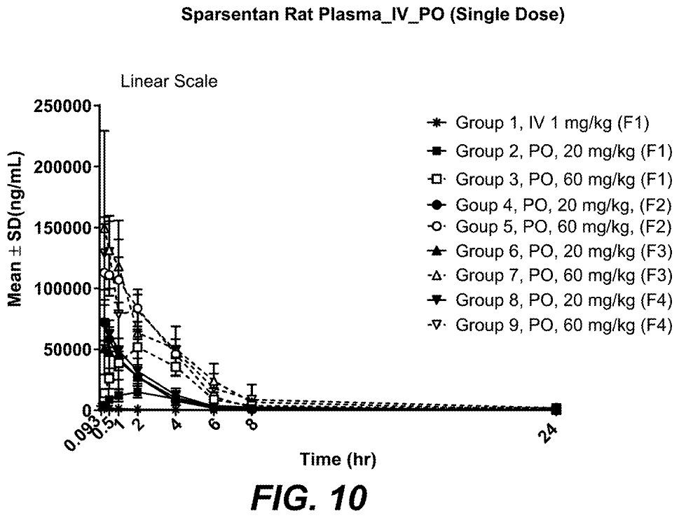

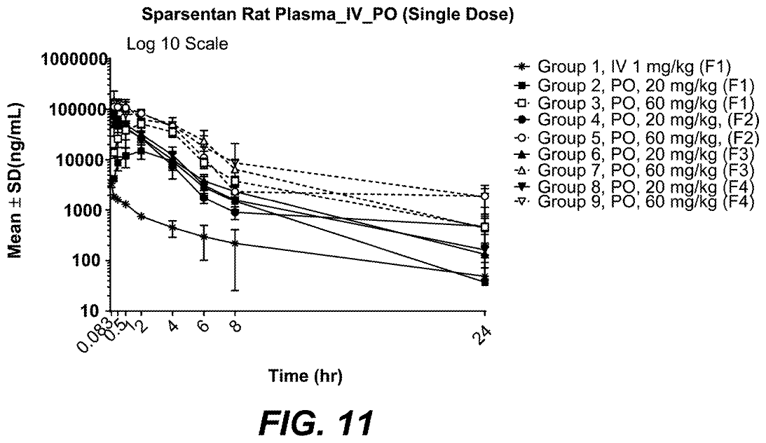

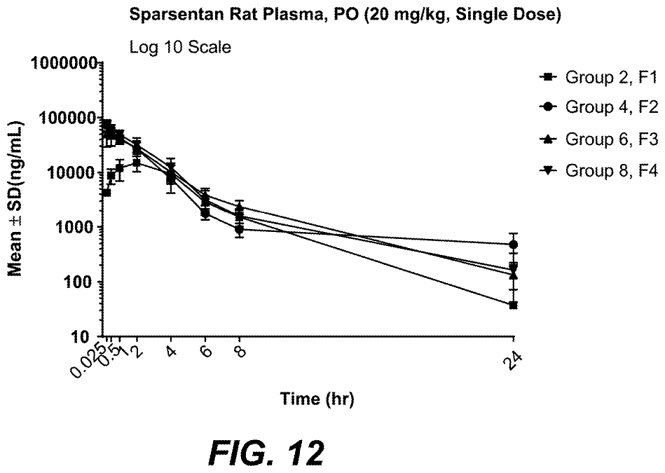

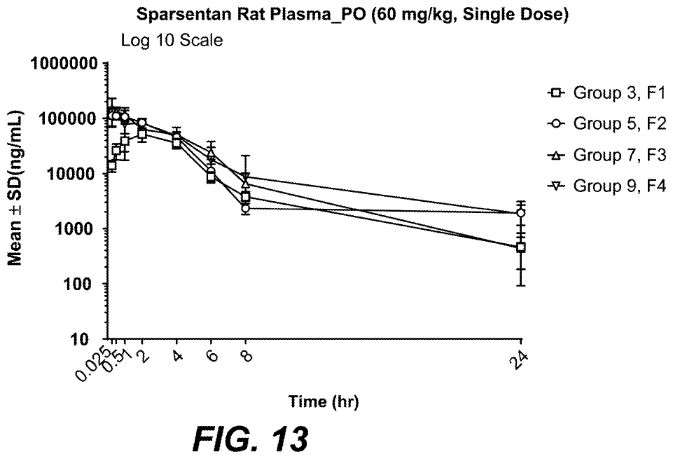

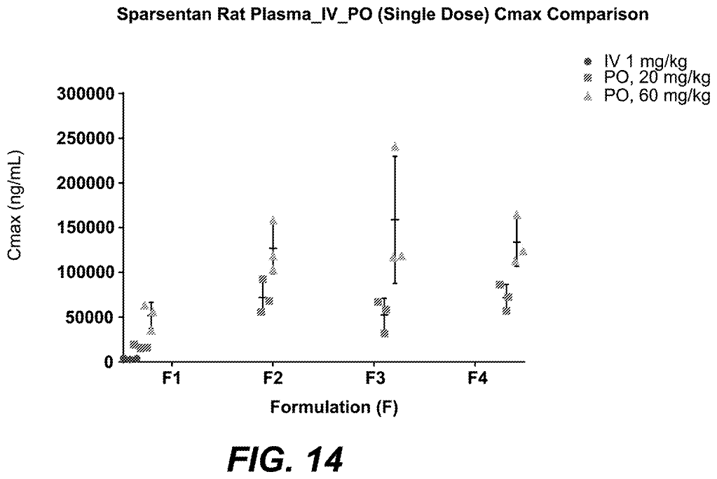

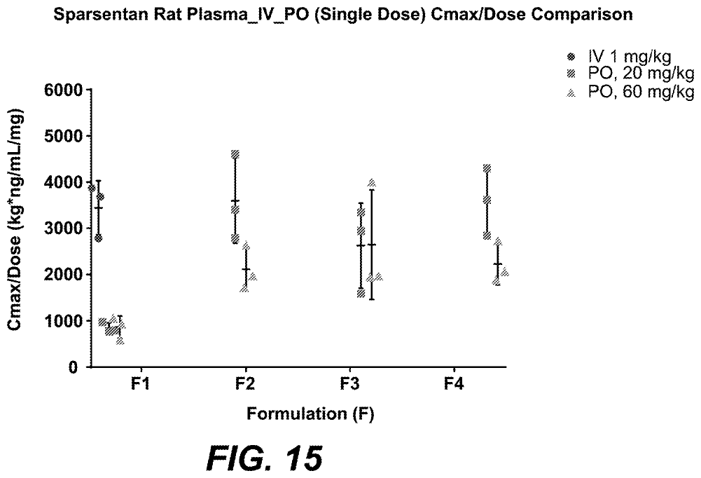

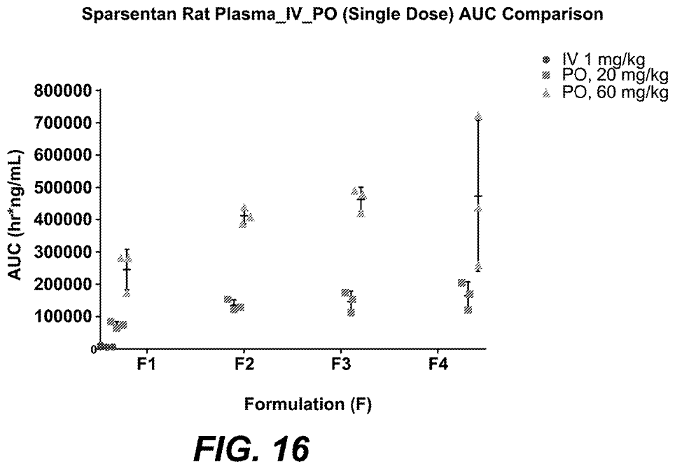

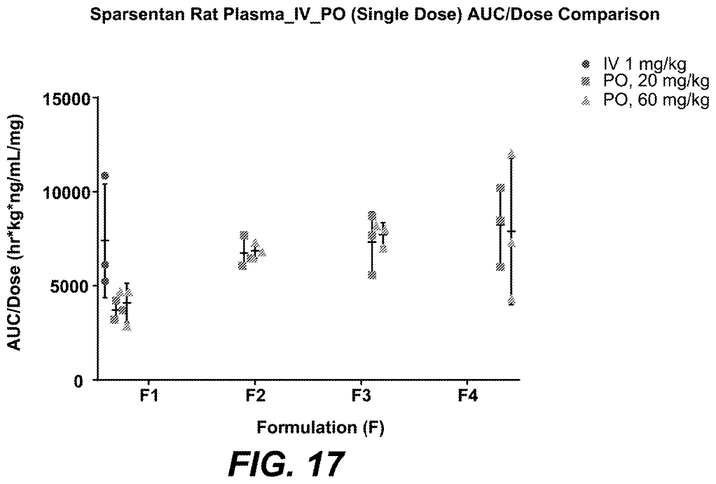

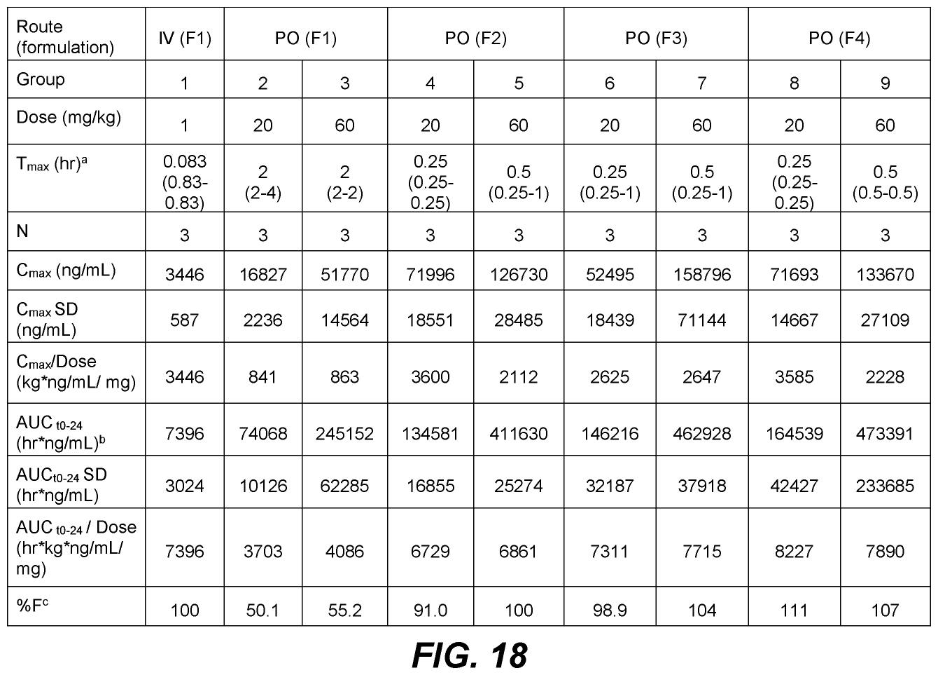

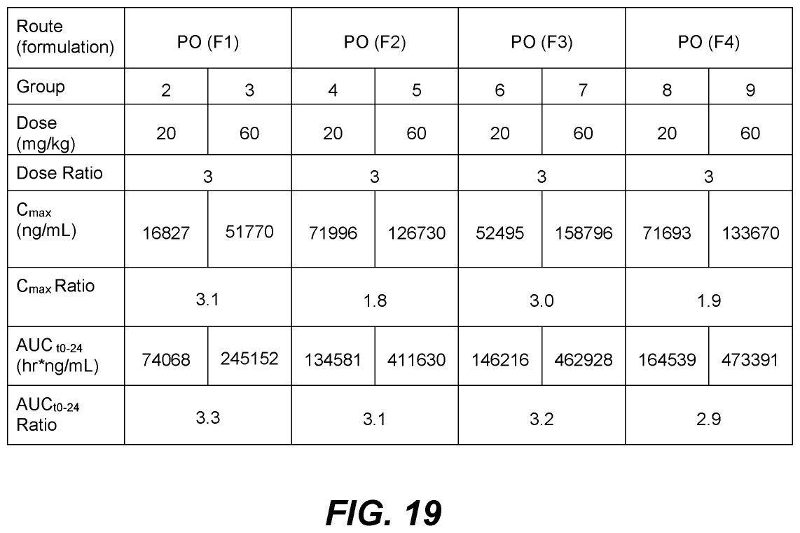

The results are shown in FIGS. 10-19. Sparsentan exposure measured by Cₘₐₓ and AUC were similar for 50:50 Sp:PVP-VA SDD, 50:50 Sp:HPMC E3LV SDD, and 50:50 Sp:HPMCAS-H SDD at 20 mg/kg or 60 mg/kg doses, and were each approximately double the values for crystalline sparsentan. For oral doses, Cₘₐₓ value increased with increasing dose in an approximately dose proportional manner for crystalline sparsentan and 50:50 Sp:HPMC E3LV SDD and in a less than dose proportional manner for 50:50 Sp:PVP-VA SDD and 50:50 Sp:HPMCAS-H SDD; however, AUC values increased with increasing dose in an approximately dose proportional manner for all four formulations. In general, the SDDs provided at an oral dose of 20 or 60 mg/kg provided better exposure than crystalline sparsentan at the same oral doses. Based on the dose-normalized AUC, the % F ranged from 91% to 100% for oral dose 50:50 Sp:PVP-VA SDD and 50:50 Sp:HPMC E3LV SDD (Groups 4-6) and ranged from 104% to 111% for oral dose 50:50 Sp:HPMC E3LV SDD and 50:50 Sp:HPMCAS-H SDD (Groups 7-9) when compared to the IV dose crystalline sparsentan. The variability observed in the oral doses may have been due to dose formulation homogeneity issues occurring during the in-life process. The % F for oral dose crystalline sparsentan was 50.1% to 55.2% for Groups 2 and 3, respectively, compared to IV dose crystalline sparsentan.

### Example 5

### Modified Release Sparsentan Formulations

The pharmacokinetics of crystalline and amorphous forms of sparsentan were further investigated.

Table 12 describes the composition of each of the formulations investigated. Six sparsentan formulations were prepared: crystalline sparsentan (“Crystalline Sp”, #1); crystalline sparsentan dosed BID (twice a day) (“Crystalline Sp BID”, #2); slow release spray dried dispersion particles formed from a 25:37.5:37.5 mixture of crystalline sparsentan, Eudragit RL, and Eudragit RS and further formulated with additional excipients (“Slow release crystalline Sp, with SLS”, #3); slow release spray dried dispersion particles formed from a 25:37.5:37.5 mixture of amorphous sparsentan, Eudragit RL, and Eudragit RS and further formulated with additional excipients (“Slow release amorphous Sp, with SLS”, #4); spray dried dispersion particles formed from a 25:65:10 mixture of sparsentan, hypromellose acetate succinate HG, and Vitamin E TPGS (“Amorphous/HPMCAS-H/TPGS Sp, without SLS”, #5); and slow release spray dried dispersion particles formed from a 25:37.5:37.5 mixture of amorphous sparsentan, Eudragit RL, and Eudragit RS and further formulated with additional excipients but omitting the surfactant (sodium lauryl sulfate, SLS) (“Slow release amorphous Sp, without SLS”, #6).

TABLE 12
Spray drying process parameters for preparing sparsentan
formulations of Example 5.

| | Formulation | | |
|---|---|---|---|
| | 5 | 4-6 | 3 |
| | 25:65:10 | 25:37.5:37.5 | 25:37.5:37.5 |
| Spray drying | Sp:HPMCAS-H: | Sp:Eudragit RL: | Sp:Eudragit RL: |
| composition | Vit E TPGS | Eudragit RS | Eudragit RS |
| Batch size, total solids | 60 | 60 | 60 |
| (g) | | | |
| API lot | C14052048-RF-18602 | | |
| SDD lot numbers | R6-1141-1 | R6-1141-5 | R6-1141-9 |
| Date of manufacture | 1 Nov. 2019 | 4 Nov. 2019 | 11 Nov. 2019 |
| Spray solvent | Acetone | | 50:50 IPA: Water |
| Spray solution | 10 | 5 | 3 |
| Composition (wt % total | | | |
| solids) | | | |
| Nozzle type | 0.7 mm liquid tip, 1.5 mm air cap | | Two-fluid |
| Drying gas mode | Recycle | | |
| Cyclone type | High | High efficiency/ | Standard |
| | efficiency | Standard | |
| Solution flow rate | 18-20 | 14-26 | 5-10 |
| (g/min) | | | |
| Atomization pressure | 28 | | |
| (psi) | | | |
| Inlet temperature (° C.) | 118-145 | 82-102 | 210-220 |
| Outlet temperature (° C.) | 42-45 | 44-47 | 89-93 |
| Condenser temperature | −20 | −20 | 0 |
| (° C.) | | | |
| Secondary drying | 40° C./24 hr | | |
| temperature/time | | | |
| Wet SDD yield (%) | 77.8 | 56 | 34 |
| Dry SDD yield (%) | 76.05 | 55 | 34 |

The spray-dried formulations were further formulated by blending with intragranular excipients, de-lumping via #30 mesh sieve, granulating via slug and mill process, adding extragranular excipients, and blending in a Turbula Blender. The intragranular and extragranular components are shown in Table 13.

TABLE 13
Intragranular and extragranular components included in Formulations
3-6 of Example 5.

| | | Formulation | |
|---|---|---|---|
| | | #3, #4 | #5, #6 |
| Component | | % w/w | % w/w |
| Intragranular | Sparsentan SDD | 50 | 50 |
| | Microcrystalline Cellulose (Avicel | 10.25 | 12.75 |
| | PH-102) | | |
| | Mannitol (Pearlitol 100 SD) | 10.25 | 12.75 |
| | Croscarmellose Sodium (Ac-Di- | 4 | 4 |
| | Sol) | | |
| | Sodium Lauryl Sulfate (SLS) | 5 | 0 |
| | Colloidal Silica (Cab-O-Sil) | 1 | 1 |
| | Magnesium Stearate | 0.5 | 0.5 |
| | Intragranular Total (percent) | 81 | 81 |
| Extragranular | Microcrystalline Cellulose (Avicel | 16.5 | 16.5 |
| | PH-200) | | |
| | Croscarmellose Sodium (Ac-Di- | 2 | 2 |
| | Sol) | | |
| | Magnesium Stearate | 0.5 | 0.5 |
| | Extragranular Total (percent) | 19 | 19 |

| | | |
|---|---|---|
| Total (percent): | 100 | 100 |

Sparsentan was administered orally (“PO”) to male Sprague Dawley rats in a single or twice-a-day dose, as described in Table 14. Formulations were administered in a vehicle comprised of a mixture of 0.5% methylcellulose 4000 cps and 0.25% Tween 80 in distilled water. All animals were fasted overnight through approximately 4 hours post-dose. Animals receiving Formulation #2 were not fasted for the second dose administration.

TABLE 14
Animal dosing levels for Formulations 1-6 of Example 5.

| | | | Dose | Dose |
|---|---|---|---|---|
| Formulation | Test Article | n | Route | (mg/kg) |
| 1 | Crystalline Sp | 3 | PO | 60 |
| 2 | Crystalline Sp BID | 3 | PO^{a} | 30 × 2 |
| 3 | Slow release crystalline | 3 | PO | 38 |
| | Sp, with SLS | | | |
| 4 | Slow release amorphous | 3 | PO | 29 |
| | Sp, with SLS | | | |
| 5 | Amorphous/HPMCAS- | 3 | PO^{b} | 30 |
| | H/TPGS Sp, without | | | |
| | SLS | | | |
| 6 | Slow release amorphous | 3 | PO | 29 |
| | Sp, without SLS | | | |

^{a}Animals were dosed twice on Day 1, with approximately 8 hours (±10 minutes) between doses.
^{b}The formulation pH was adjusted to pH 4.0 prior to dose administration.

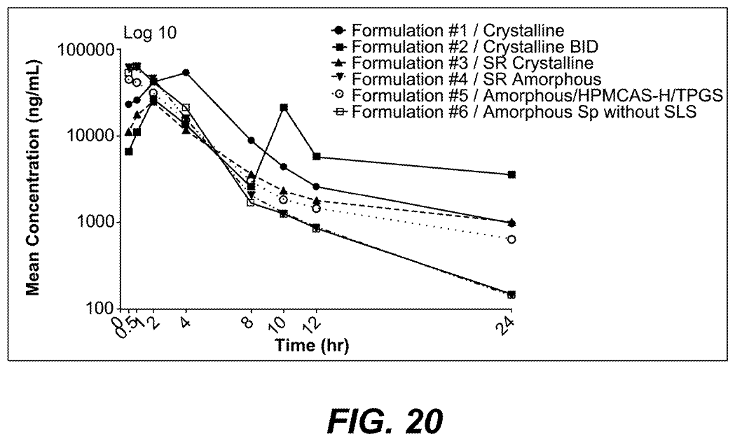

Blood (approximately 0.3 mL) was collected from a jugular vein from each animal via syringe and needle and transferred into tubes containing K2EDTA at approximately 0.5, 1, 2, 4, 8 (prior to second dose for Group 2), 10, 12, and 24 hours post-dose. Another vein may be used as an alternative blood collection site. For each group, the following PK parameters were determined: maximum observed plasma concentration (Cₘₐₓ), time of maximum observed plasma concentration (Tₘₐₓ), and area under the plasma concentration-time curve (AUC). AUC from time 0 to 24 hours (AUC_{0-24 hr}) was calculated for all groups with at least three consecutive quantifiable concentrations. The PK parameters are shown in Table 15 and FIG. 20.

TABLE 15
PK parameters after administration of sparsentan formulations to rats.

| | Formulation | | | | | |
|---|---|---|---|---|---|---|
| | #1 | #2^{a} | #3 | #4 | #5 | #6 |
| Dose | 60 | 60 | 38 | 28.8 | 30 | 28.6 |
| (mg/kg/day) | | | | | | |
| Tₘₐₓ (hr)^{b} | 4 | 2 | 2 | 0.5 | 0.5 | 1 |
| | (4-4) | (2-2) | (2-2) | (0.5-1) | (0.5-1) | (1-1) |
| Cₘₐₓ (ng/mL) | 54567 | 26700 | 25033 | 62400 | 45500 | 63833 |
| Cₘₐₓ/Dose | 909 | 890 | 659 | 2167 | 1517 | 2232 |
| (kg*ng/ | | | | | | |
| mL/mg) | | | | | | |
| AUC_{0.5-24 hr} | 311335 | 204206 | 123565 | 195480 | 163321 | 203728 |
| (hr*ng/mL) | | | | | | |
| AUC_{0.5-24 hr}/Dose | 5189 | 3403 | 3252 | 6788 | 5444 | 7123 |
| (hr*kg*ng | | | | | | |
| /mL/mg) | | | | | | |

^{a}30 mg/kg/dose, 60 mg/kg/day with approximately 8 hr between doses. Tmax and Cmax were determined from first dose. AUC was determined from the total dose.
^{b}Median (min-max) value.

All of the U.S. patents, U.S. patent application publications, U.S. patent applications, foreign patents, foreign patent applications, and non-patent publications referred to in this specification or listed in the Application Data Sheet, including U.S. Provisional Patent Application No. 62/783,947 filed Dec. 21, 2018, are incorporated herein by reference, in their entirety to the extent not inconsistent with the present description, unless otherwise stated. Aspects of the embodiments can be modified, if necessary, to employ concepts of the various patents, applications, and publications to provide yet further embodiments.

While specific embodiments have been illustrated and described, it will be readily appreciated that the various embodiments described above can be combined to provide further embodiments, and that various changes can be made therein without departing from the spirit and scope of the invention.

These and other changes can be made to the embodiments in light of the above-detailed description.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. An amorphous form of a compound having structure (I), 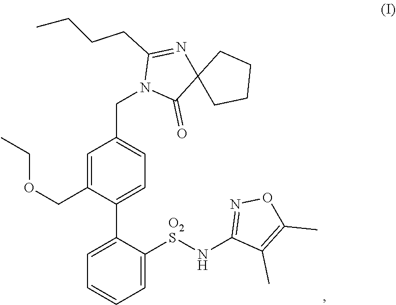 [SMILES: CCCCC1=NC2(CCCC2)C(=O)N1Cc1ccc(-c2ccccc2S(=O)(=O)Nc2noc(C)c2C)c(COCC)c1]
or a pharmaceutically acceptable salt thereof.

2. A pharmaceutical composition comprising (1) an amorphous compound according to claim 1, and (2) a pharmaceutically acceptable excipient.

3. The pharmaceutical composition according to claim 2, wherein at least 50% of the compound according to claim 1 by weight percent is present in an amorphous form.

4. The pharmaceutical composition according to claim 2, wherein at least 60% of the compound according to claim 1 by weight percent is present in an amorphous form.

5. The pharmaceutical composition according to claim 2, wherein at least 70% of the compound according to claim 1 by weight percent is present in an amorphous form.

6. The pharmaceutical composition according to claim 2, wherein at least 80% of the compound according to claim 1 by weight percent is present in an amorphous form.

7. The pharmaceutical composition according to claim 2, wherein at least 90% of the compound according to claim 1 by weight percent is present in an amorphous form.

8. The pharmaceutical composition according to claim 2, wherein at least 95% of the compound according to claim 1 by weight percent is present in an amorphous form.

9. The pharmaceutical composition according to claim 2, wherein at least 98% of the compound according to claim 1 by weight percent is present in an amorphous form.

10. The pharmaceutical composition according to claim 2, wherein at least 99% of the compound according to claim 1 by weight percent is present in an amorphous form.

11. The pharmaceutical composition according to any one of claims 2-10, further comprising a pharmaceutically acceptable polymer.

12. The pharmaceutical composition of claim 11, wherein said polymer comprises hydroxypropyl methylcellulose (hypromellose); hydroxypropyl methylcellulose for HME; hypromellose acetate succinate LG; hypromellose acetate succinate MG; hypromellose acetate succinate HG; hypromellose acetate succinate 716; hypromellose acetate succinate 912; hypromellose acetate succinate 126; polyvinylpyrrolidone/vinyl acetate copolymer; or polyvinyl caprolactam-polyvinyl acetate-polyethylene glycol graft copolymer.

13. The pharmaceutical composition of claim 11 or claim 12, wherein the weight ratio of said amorphous compound having structure (I), or pharmaceutically acceptable salt thereof, to said polymer is at least 95:5, 90:10, 85:15, 80:20, 75:25, 70:30, 65:35, 60:40, 55:45, 50:50, 45:55, 40:60, 35:65, 30:70, or 25:75.

14. The pharmaceutical composition of claim 11 or claim 12, wherein the weight ratio of said amorphous compound having structure (I), or pharmaceutically acceptable salt thereof, to said polymer is from 25:75 to 95:5.

15. The pharmaceutical composition of any one of claims 11-14, wherein said amorphous compound and said polymer have been formed by spray drying a dispersion comprising said compound and said polymer.

16. The pharmaceutical composition according to any one of claims 2-15, wherein said amorphous compound has structure (I).

17. A method of treating a kidney disease or disorder in a subject in need thereof, comprising administering to said subject the compound of claim 1 or the pharmaceutical composition of any one of claims 2-16.

18. The compound of claim 1 or the pharmaceutical composition of any one of claims 2-16 for use in treating a kidney disease or disorder in a subject in need thereof.

19. The use of the compound of claim 1 or the pharmaceutical composition of any one of claims 2-16 for the manufacture of a medicament for the treatment of a kidney disease or disorder.

20. The method of claim 17, the compound or pharmaceutical composition for use of claim 18, or the use of claim 19, wherein the kidney disease or disorder is focal segmental glomerulosclerosis (FSGS).

21. The method of claim 17, the compound or pharmaceutical composition for use of claim 18, or the use of claim 19, wherein the kidney disease or disorder is IgA nephropathy (IgAN).

22. The method of claim 17, the compound or pharmaceutical composition for use of claim 18, or the use of claim 19, wherein the kidney disease or disorder is diabetic nephropathy.

23. The method of claim 17, the compound or pharmaceutical composition for use of claim 18, or the use of claim 19, wherein the kidney disease or disorder is idiopathic membranous nephropathy (IMN).

24. The method of claim 17, the compound or pharmaceutical composition for use of claim 18, or the use of claim 19, wherein the kidney disease or disorder is Alport syndrome.

25. A method of treating hearing loss associated with Alport syndrome in a subject in need thereof, comprising administering to said subject a compound of claim 1 or a pharmaceutical composition of any one of claims 2-16.

26. The compound of claim 1 or the pharmaceutical composition of any one of claims 2-16 for use in treating of hearing loss associated with Alport syndrome in a subject in need thereof.

27. The use of the compound of claim 1 or the pharmaceutical composition of any one of claims 2-16 for the manufacture of a medicament for the treatment of hearing loss associated with Alport syndrome.

28. The method of any one of claims 17 and 20-25, the compound or pharmaceutical composition for use of any one of claims 18, 20-24, and 26, or the use of any one of claims 19-24 and 27, wherein the amount of said amorphous compound having structure (I), or pharmaceutically acceptable salt thereof, administered to said subject is from about 50 mg/day to about 1000 mg/day.

29. The method of any one of claims 17 and 20-25, the compound or pharmaceutical composition for use of any one of claims 18, 20-24, and 26, or the use of any one of claims 19-24 and 27, wherein the amount of said amorphous compound having structure (I), or pharmaceutically acceptable salt thereof, administered to said subject is from about 50 mg/day to about 800 mg/day.

30. The method of any one of claims 17 and 20-25, the compound or pharmaceutical composition for use of any one of claims 18, 20-24, and 26, or the use of any one of claims 19-24 and 27, wherein the amount of said amorphous compound having structure (I), or pharmaceutically acceptable salt thereof, administered to said subject is from about 200 mg/day to about 400 mg/day.

31. The method of any one of claims 17 and 20-25, the compound or pharmaceutical composition for use of any one of claims 18, 20-24, and 26, or the use of any one of claims 19-24 and 27, wherein the amount of said amorphous compound having structure (I), or pharmaceutically acceptable salt thereof, administered to said subject is from about 400 mg/day to about 800 mg/day.

32. The method of any one of claims 17 and 20-25, the compound or pharmaceutical composition for use of any one of claims 18, 20-24, and 26, or the use of any one of claims 19-24 and 27, wherein the amount of said amorphous compound having structure (I), or pharmaceutically acceptable salt thereof, administered to said subject is about 50 mg/day.

33. The method of any one of claims 17 and 20-25, the compound or pharmaceutical composition for use of any one of claims 18, 20-24, and 26, or the use of any one of claims 19-24 and 27, wherein the amount of said amorphous compound having structure (I), or pharmaceutically acceptable salt thereof, administered to said subject is about 100 mg/day.

34. The method of any one of claims 17 and 20-25, the compound or pharmaceutical composition for use of any one of claims 18, 20-24, and 26, or the use of any one of claims 19-24 and 27, wherein the amount of said amorphous compound having structure (I), or pharmaceutically acceptable salt thereof, administered to said subject is about 200 mg/day.

35. The method of any one of claims 17 and 20-25, the compound or pharmaceutical composition for use of any one of claims 18, 20-24, and 26, or the use of any one of claims 19-24 and 27, wherein the amount of said amorphous compound having structure (I), or pharmaceutically acceptable salt thereof, administered to said subject is about 400 mg/day.

36. The method of any one of claims 17 and 20-25, the compound or pharmaceutical composition for use of any one of claims 18, 20-24, and 26, or the use of any one of claims 19-24 and 27, wherein the amount of said amorphous compound having structure (I), or pharmaceutically acceptable salt thereof, administered to said subject is about 800 mg/day.

37. The method of any one of claims 17, 20-25, and 28-36, the compound or pharmaceutical composition for use of any one of claims 18, 20-24, 26, and 28-36, or the use of any one of claims 19-24 and 27-36, wherein said subject is administered one or more additional therapeutic agents.

38. The method of any one of claims 17, 20-25, and 28-37, the compound or pharmaceutical composition for use of any one of claims 18, 20-24, 26, and 28-37, or the use of any one of claims 19-24 and 27-37, wherein said subject is an adult.

39. The method of any one of claims 17, 20-25, and 28-37, the compound or pharmaceutical composition for use of any one of claims 18, 20-24, 26, and 28-37, or the use of any one of claims 19-24 and 27-37, wherein said subject is 18 years old or younger.
